SEQUENCE LISTING (136 sequences)

SEQ ID NO: 1 - protein, 308 aa, Nipponia nippon
IALDKMEDLSLKETVVSRAEICEVEGIPFTKPICSTWDQVWKFKARPDDLLIATYTKAGTTWTQEIVDMIQQNGDVEKCRRATTYRRHPFLEWSIQEPPAASYSGLELAEAMPSPRTIKTHLPVQLLPPSFWEQNCKIIYVARNAKDNLVSYYHFHRMSKEMPDPGTWEEFMEKFMTGKVLWGSWYDHVKGWWKAKDRHRILYLFYEDMKENPKQEIQKILKFLEKDVNQEVLNKILHNTSFEIMKDNPMTNYTTEFQGIMDHSISPFMRKGVVGDWKNYFTVAQNEKFDEDYKKKMADTSLVFRTEL

SEQ ID NO: 2 - DNA, 28 bp, synthetic construct
  source 1..28: mol_type = other DNA
cataaaatcacctcaacctctagatacc

SEQ ID NO: 3 - DNA, 22 bp, synthetic construct
  source 1..22: mol_type = other DNA
taagtcgaccgatgcccttgag

SEQ ID NO: 4 - DNA, 55 bp, synthetic construct
  source 1..55: mol_type = other DNA
gaattcattaaagaggagaaattacatatggaattgattcaagatacgagccgcc

SEQ ID NO: 5 - DNA, 53 bp, synthetic construct
  source 1..53: mol_type = other DNA
gctctcaagggcatcggtcgacttatcacagctcactacgaaagctaagcgag

SEQ ID NO: 6 - DNA, 55 bp, synthetic construct
  source 1..55: mol_type = other DNA
gaattcattaaagaggagaaattacatatggagttctctcgccctccacttgtgc

SEQ ID NO: 7 - DNA, 53 bp, synthetic construct
  source 1..53: mol_type = other DNA
gctctcaagggcatcggtcgacttatcacaattcgcaacgaaacttgaaatcg

SEQ ID NO: 8 - DNA, 55 bp, synthetic construct
  source 1..55: mol_type = other DNA
gaattcattaaagaggagaaattacatatggaacttatccaggatacctcccgtc

SEQ ID NO: 9 - DNA, 54 bp, synthetic construct
  source 1..54: mol_type = other DNA
gctctcaagggcatcggtcgacttatcaaagctccgagcgaaacgacagtgaac

SEQ ID NO: 10 - DNA, 54 bp, synthetic construct
  source 1..54: mol_type = other DNA
gaattcattaaagaggagaaattacatatggctctggacaagatggaaaacttg

SEQ ID NO: 11 - DNA, 54 bp, synthetic construct
  source 1..54: mol_type = other DNA
gctctcaagggcatcggtcgacttatcaaagttccatacggaaaaccaagctag

SEQ ID NO: 12 - DNA, 51 bp, synthetic construct
  source 1..51: mol_type = other DNA
gtatctagaggttgaggtgattttatggccttaacatctgaccttggtaag

SEQ ID NO: 13 - DNA, 51 bp, synthetic construct
  source 1..51: mol_type = other DNA
gctctcaagggcatcggtcgacttatcagagttccatgcagaagttgatgc

SEQ ID NO: 14 - DNA, 52 bp, synthetic construct
  source 1..52: mol_type = other DNA
gtatctagaggttgaggtgattttatggccttaacttcagagttagggaaac

SEQ ID NO: 15 - DNA, 56 bp, synthetic construct
  source 1..56: mol_type = other DNA
gctctcaagggcatcggtcgacttatcacagctccatacagaagttaatacttgtg

SEQ ID NO: 16 - DNA, 54 bp, synthetic construct
  source 1..54: mol_type = other DNA
gtatctagaggttgaggtgattttatggctcaggttcctgaattatcgaaaccg

SEQ ID NO: 17 - DNA, 57 bp, synthetic construct
  source 1..57: mol_type = other DNA
gctctcaagggcatcggtcgacttatcacagtttcatacaaaagttaatcgaagtgc

SEQ ID NO: 18 - DNA, 54 bp, synthetic construct
  source 1..54: mol_type = other DNA
gtatctagaggttgaggtgattttatgcttttaatcagcacatatgcaaaagcg

SEQ ID NO: 19 - DNA, 57 bp, synthetic construct
  source 1..57: mol_type = other DNA
gctctcaagggcatcggtcgacttatcattacagttctgtacggaaaacaagtgaag

SEQ ID NO: 20 - DNA, 48 bp, synthetic construct
  source 1..48: mol_type = other DNA
gtatctagaggttgaggtgattttatgattgagcaaaacggggacgtg

SEQ ID NO: 21 - DNA, 56 bp, synthetic construct
  source 1..56: mol_type = other DNA
gctctcaagggcatcggtcgacttatcacagttccatacaaaaattgatcgaggtc

SEQ ID NO: 22 - DNA, 52 bp, synthetic construct
  source 1..52: mol_type = other DNA
gtatctagaggttgaggtgattttatggcattaacatctgagcttggtaagc

SEQ ID NO: 23 - DNA, 57 bp, synthetic construct
  source 1..57: mol_type = other DNA
gctctcaagggcatcggtcgacttatcacagttccatgcagaagttaatacttgtcc

SEQ ID NO: 24 - DNA, 49 bp, synthetic construct
  source 1..49: mol_type = other DNA
gtatctagaggttgaggtgattttatggatatgattgagcagaacggcg

SEQ ID NO: 25 - DNA, 58 bp, synthetic construct
  source 1..58: mol_type = other DNA
gctctcaagggcatcggtcgacttatcacagctccatgcagaaattaattgaagtacc

SEQ ID NO: 26 - DNA, 46 bp, synthetic construct
  source 1..46: mol_type = other DNA
gtatctagaggttgaggtgattttatggcgttaacctcggacttgg

SEQ ID NO: 27 - DNA, 57 bp, synthetic construct
  source 1..57: mol_type = other DNA
gctctcaagggcatcggtcgacttatcacagttccatacaaaagttaatagccgtcc

SEQ ID NO: 28 - DNA, 52 bp, synthetic construct
  source 1..52: mol_type = other DNA
gtatctagaggttgaggtgattttatggctctgacttctgaactggggaaac

SEQ ID NO: 29 - DNA, 57 bp, synthetic construct
  source 1..57: mol_type = other DNA
gctctcaagggcatcggtcgacttatcacagctccatacaaaaattaattgcagtgc

SEQ ID NO: 30 - DNA, 51 bp, synthetic construct
  source 1..51: mol_type = other DNA
gtatctagaggttgaggtgattttatggcattaacaagcgagttggggaag

SEQ ID NO: 31 - DNA, 56 bp, synthetic construct
  source 1..56: mol_type = other DNA
gctctcaagggcatcggtcgacttatcacagctccatgcaaaagttaatgcttgtg

SEQ ID NO: 32 - DNA, 48 bp, synthetic construct
  source 1..48: mol_type = other DNA
gtatctagaggttgaggtgattttatggctctcgacaaaatggaggac

SEQ ID NO: 33 - DNA, 50 bp, synthetic construct
  source 1..50: mol_type = other DNA
gctctcaagggcatcggtcgacttatcacagctcagtgcgaaagaccaac

SEQ ID NO: 34 - DNA, 49 bp, synthetic construct
  source 1..49: mol_type = other DNA
gtatctagaggttgaggtgattttatggcccttatcactgcaggtactc

SEQ ID NO: 35 - DNA, 57 bp, synthetic construct
  source 1..57: mol_type = other DNA
gctctcaagggcatcggtcgacttatcacagttctgtgcaaaaattgatgctggtac

SEQ ID NO: 36 - DNA, 53 bp, synthetic construct
  source 1..53: mol_type = other DNA
gtatctagaggttgaggtgattttaacatggagcttatcaaagacatttcgcg

SEQ ID NO: 37 - DNA, 50 bp, synthetic construct
  source 1..50: mol_type = other DNA
gctctcaagggcatcggtcgacttatcacaggttgcaccgaaatttgagg

SEQ ID NO: 38 - DNA, 48 bp, synthetic construct
  source 1..48: mol_type = other DNA
gtatctagaggttgaggtgattttatggcgcaaaatccaagcaacatg

SEQ ID NO: 39 - DNA, 59 bp, synthetic construct
  source 1..59: mol_type = other DNA
gctctcaagggcatcggtcgacttatcaaatttgacaacgaaatgtgaaatcgcaaccg

SEQ ID NO: 40 - DNA, 44 bp, synthetic construct
  source 1..44: mol_type = other DNA
gtatctagaggttgaggtgattttatggaaccactgcggaagcc

SEQ ID NO: 41 - DNA, 53 bp, synthetic construct
  source 1..53: mol_type = other DNA
gctctcaagggcatcggtcgacttatcaaatttggcaccgaaaagtgaagtcg

SEQ ID NO: 42 - DNA, 48 bp, synthetic construct
  source 1..48: mol_type = other DNA
gtatctagaggttgaggtgattttatggcccagaacccatctaacatg

SEQ ID NO: 43 - DNA, 54 bp, synthetic construct
  source 1..54: mol_type = other DNA
gctctcaagggcatcggtcgacttatcaaatttgacaccggaatgtgaagtcac

SEQ ID NO: 44 - DNA, 44 bp, synthetic construct
  source 1..44: mol_type = other DNA
gtatctagaggttgaggtgattttatggcgggcgaagatcacac

SEQ ID NO: 45 - DNA, 54 bp, synthetic construct
  source 1..54: mol_type = other DNA
gctctcaagggcatcggtcgacttatcaaatttcagtgcggaatgtaagggtac

SEQ ID NO: 46 - DNA, 45 bp, synthetic construct
  source 1..45: mol_type = other DNA
gtatctagaggttgaggtgattttatgcggaaacctgagctggag

SEQ ID NO: 47 - DNA, 47 bp, synthetic construct
  source 1..47: mol_type = other DNA
gctctcaagggcatcggtcgacttatcacagttcgaggcaaaaccgg

SEQ ID NO: 48 - DNA, 46 bp, synthetic construct
  source 1..46: mol_type = other DNA
gtatctagaggttgaggtgattttatggccctcactagcgaacttg

SEQ ID NO: 49 - DNA, 56 bp, synthetic construct
  source 1..56: mol_type = other DNA
gctctcaagggcatcggtcgacttatcacagttccatacagaagttaatggacgtg

SEQ ID NO: 50 - DNA, 47 bp, synthetic construct
  source 1..47: mol_type = other DNA
gtatctagaggttgaggtgattttatggctttaactaccgcgggtac

SEQ ID NO: 51 - DNA, 56 bp, synthetic construct
  source 1..56: mol_type = other DNA
gctctcaagggcatcggtcgacttatcacagttcggtgcagaagttaatacttgtc

SEQ ID NO: 52 - DNA, 53 bp, synthetic construct
  source 1..53: mol_type = other DNA
gtatctagaggttgaggtgattttatggctttggataagatggaagacctctc

SEQ ID NO: 53 - DNA, 50 bp, synthetic construct
  source 1..50: mol_type = other DNA
gctctcaagggcatcggtcgacttatcagagttctgtgcggaagactacg

SEQ ID NO: 54 - DNA, 48 bp, synthetic construct
  source 1..48: mol_type = other DNA
gtatctagaggttgaggtgattttatggcgctggacaaaatgaaggac

SEQ ID NO: 55 - DNA, 53 bp, synthetic construct
  source 1..53: mol_type = other DNA
gctctcaagggcatcggtcgacttatcactccatacgaaagaccagagatgtg

SEQ ID NO: 56 - DNA, 54 bp, synthetic construct
  source 1..54: mol_type = other DNA
gtatctagaggttgaggtgattttatgcgtatggaagatctttcgctgaaatac

SEQ ID NO: 57 - DNA, 51 bp, synthetic construct
  source 1..51: mol_type = other DNA
gctctcaagggcatcggtcgacttatcacaattccatgcggaagactaacg

SEQ ID NO: 58 - DNA, 52 bp, synthetic construct
  source 1..52: mol_type = other DNA
gtatctagaggttgaggtgattttatgtgcaatgtgttccagatcactaccg

SEQ ID NO: 59 - DNA, 47 bp, synthetic construct
  source 1..47: mol_type = other DNA
gctctcaagggcatcggtcgacttatcacaactctgcccgaaacacc

SEQ ID NO: 60 - DNA, 54 bp, synthetic construct
  source 1..54: mol_type = other DNA
gtatctagaggttgaggtgattttatggtagacaaaatgaaagacctctcactc

SEQ ID NO: 61 - DNA, 53 bp, synthetic construct
  source 1..53: mol_type = other DNA
gctctcaagggcatcggtcgacttatcataattccatgcggaaaaccaacgag

SEQ ID NO: 62 - DNA, 49 bp, synthetic construct
  source 1..49: mol_type = other DNA
gtatctagaggttgaggtgattttatgctggccatggacaagatgaaag

SEQ ID NO: 63 - DNA, 51 bp, synthetic construct
  source 1..51: mol_type = other DNA
gctctcaagggcatcggtcgacttatcacaattccatacggaagacgaggc

SEQ ID NO: 64 - DNA, 49 bp, synthetic construct
  source 1..49: mol_type = other DNA
gtatctagaggttgaggtgattttatgtctggcactacatggactcagg

SEQ ID NO: 65 - DNA, 51 bp, synthetic construct
  source 1..51: mol_type = other DNA
gctctcaagggcatcggtcgacttatcaaagttccgtccggaaaacaagtg

SEQ ID NO: 66 - DNA, 19 bp, synthetic construct
  source 1..19: mol_type = other DNA
gcatgctcgagcagctcag

SEQ ID NO: 67 - DNA, 33 bp, synthetic construct
  source 1..33: mol_type = other DNA
agatctaattcctcctgttagcccaaaaaaacg

SEQ ID NO: 68 - DNA, 49 bp, synthetic construct
  source 1..49: mol_type = other DNA
tgggctaacaggaggaattagatctatggctctcgacaaaatggaggac

SEQ ID NO: 69 - DNA, 48 bp, synthetic construct
  source 1..48: mol_type = other DNA
cgaccctgagctgctcgagcatgcagaagacagtcataagtgcggcga

SEQ ID NO: 70 - DNA, 33 bp, synthetic construct
  source 1..33: mol_type = other DNA
atgggattcctcaaagcgtaaacaacgtataac

SEQ ID NO: 71 - DNA, 48 bp, synthetic construct
  source 1..48: mol_type = other DNA
gtttacgctttgaggaatcccatatggatcaaatacgacttactcacc

SEQ ID NO: 72 - DNA, 61 bp, synthetic construct
  source 1..61: mol_type = other DNA
tgcgcgaccctgagctgctcgagcatgctcaggatctgataatatcgttctgtctcaacag

SEQ ID NO: 73 - DNA, 33 bp, synthetic construct
  source 1..33: mol_type = other DNA
tcaggatctgataatatcgttctgtctcaacag

SEQ ID NO: 74 - DNA, 53 bp, synthetic construct
  source 1..53: mol_type = other DNA
gacagaacgatattatcagatcctgataagttaacaccgctcacagagacgag

SEQ ID NO: 75 - DNA, 55 bp, synthetic construct
  source 1..55: mol_type = other DNA
cgaccctgagctgctcgagcatgcttagtaaatagacactctgaaccccggattc

SEQ ID NO: 76 - DNA, 35 bp, synthetic construct
  source 1..35: mol_type = other DNA
gtcgaccatcatcatcatcatcattgagtttaaac

SEQ ID NO: 77 - DNA, 60 bp, synthetic construct
  source 1..60: mol_type = other DNA
tcactatagggagacccaagctggctagcgccaccatggcgagttccaatctgattaagc

SEQ ID NO: 78 - DNA, 59 bp, synthetic construct
  source 1..59: mol_type = other DNA
gagttaaagtcgacttaacgcgttgaattcttatacaggtcctttccagcaaatgagac

SEQ ID NO: 79 - DNA, 56 bp, synthetic construct
  source 1..56: mol_type = other DNA
attcattaaagaggagaaattacatatgcagcctaaggaaaaaacaaaaggtgtag

SEQ ID NO: 80 - DNA, 59 bp, synthetic construct
  source 1..59: mol_type = other DNA
gagtccaagctcagctaattaagcttatgagattcagaactgaagtcaggaatctgatc

SEQ ID NO: 81 - DNA, 47 bp, synthetic construct
  source 1..47: mol_type = other DNA
attcattaaagaggagaaattacatatgtatccagaacgggactccg

SEQ ID NO: 82 - DNA, 45 bp, synthetic construct
  source 1..45: mol_type = other DNA
gagtccaagctcagctaattaagcttcggtttgttgccccgaagg

SEQ ID NO: 83 - DNA, 24 bp, synthetic construct
  source 1..24: mol_type = other DNA
ttacgacgagtaggaggacaacgc

SEQ ID NO: 84 - DNA, 21 bp, synthetic construct
  source 1..21: mol_type = other DNA
ctaacgtccatctgacgggcg

SEQ ID NO: 85 - DNA, 29 bp, synthetic construct
  source 1..29: mol_type = other DNA
ggacgcggattaggatgaatatgaggaag

SEQ ID NO: 86 - DNA, 21 bp, synthetic construct
  source 1..21: mol_type = other DNA
ccgtcggtacgtttctgcacc

SEQ ID NO: 87 - DNA, 66 bp, synthetic construct
  source 1..66: mol_type = other DNA
ggcaaagaattgcaagtttgtacaaaaaagcaggctgccaccatggcgcttgacaaaatggaagac

SEQ ID NO: 88 - DNA, 71 bp, synthetic construct
  source 1..71: mol_type = other DNA
gcctgcacctgaggatcaccactttgtacaagaaagctgggtttaaagctctgtccgaaagacgagagaag

SEQ ID NO: 89 - DNA, 18 bp, synthetic construct
  source 1..18: mol_type = other DNA
tccgcgtccccgtcggta

SEQ ID NO: 90 - DNA, 30 bp, synthetic construct
  source 1..30: mol_type = other DNA
ttacgatgaataggaggaagacgggacgac

SEQ ID NO: 91 - DNA, 30 bp, synthetic construct
  source 1..30: mol_type = other DNA
ttaggatgaataggaggaagacgggacgac

SEQ ID NO: 92 - DNA, 22 bp, synthetic construct
  source 1..22: mol_type = other DNA
atctgacgggcggaaatgagcg

SEQ ID NO: 93 - DNA, 32 bp, synthetic construct
  source 1..32: mol_type = other DNA
ggacgttagttaggacgagtacgaggacaacg

SEQ ID NO: 94 - DNA, 32 bp, synthetic construct
  source 1..32: mol_type = other DNA
ggacgttagttaggacgagtaggaggacaacg

SEQ ID NO: 95 - DNA, 49 bp, synthetic construct
  source 1..49: mol_type = other DNA
attcattaaagaggagaaattacatatggctctcgacaaaatggaggac

SEQ ID NO: 96 - DNA, 68 bp, synthetic construct
  source 1..68: mol_type = other DNA
gagtccaagctcagctaattaagctttagtggtggtggtggtggtgcagctcagtgcgaaagaccaac

SEQ ID NO: 97 - DNA, 44 bp, synthetic construct
  source 1..44: mol_type = other DNA
cggcacccgttcctcgaatggtctgggcttgaattagcggaggc

SEQ ID NO: 98 - DNA, 20 bp, synthetic construct
  source 1..20: mol_type = other DNA
ccattcgaggaacgggtgcc

SEQ ID NO: 99 - DNA, 54 bp, synthetic construct
  source 1..54: mol_type = other DNA
gaagtcgaaggtatcccgttcgcgaagcctatttgtagtacgtgggatcaagtg

SEQ ID NO: 100 - DNA, 25 bp, synthetic construct
  source 1..25: mol_type = other DNA
gaacgggataccttcgacttcgcag

SEQ ID NO: 101 - DNA, 57 bp, synthetic construct
  source 1..57: mol_type = other DNA
gaaggtatcccgttcactaagcctgcgtgtagtacgtgggatcaagtgtggaaattc

SEQ ID NO: 102 - DNA, 25 bp, synthetic construct
  source 1..25: mol_type = other DNA
aggcttagtgaacgggataccttcg

SEQ ID NO: 103 - DNA, 44 bp, synthetic construct
  source 1..44: mol_type = other DNA
gccggcacccgttcctcgaagcgtcaatccaggagccaccggct

SEQ ID NO: 104 - DNA, 20 bp, synthetic construct
  source 1..20: mol_type = other DNA
ttcgaggaacgggtgccggc

SEQ ID NO: 105 - DNA, 49 bp, synthetic construct
  source 1..49: mol_type = other DNA
ctatcactttcaccgcatgagcaaagcgatgccagatcctgggacctgg

SEQ ID NO: 106 - DNA, 29 bp, synthetic construct
  source 1..29: mol_type = other DNA
tttgctcatgcggtgaaagtgatagtaac

SEQ ID NO: 107 - protein, 77 aa, Haemaphysalis longicornis
MQPKEKTKGVEVEGNPATLISARQMDVSYDEYEDNGPDVIPGEPAKPRGGPKNGAASGKFDQIPDFSSESHHHHHHH

SEQ ID NO: 108 - protein, 67 aa, Haemaphysalis longicornis
MYPERDSAKEGNQEQERALHVKVQKRTDGDADYDEYEEDGTTPTPDPTAPTAKPRLRGNKPHHHHHH

SEQ ID NO: 109 - protein, 92 aa, Hirudo medicinalis
MKKNIAFLLASMFVFSIATNAYAMRYTACTESGQNQCICEGNDVCGQGRNCQFDSSGKKCVEGEGTRKPQNEGQHDFDPIPEEYLSHHHHHH

SEQ ID NO: 110 - protein, 18 aa, Human immunodeficiency virus 1
KVQKEYALFYELDIVPID

SEQ ID NO: 111 - protein, 145 aa, synthetic construct
QVQLVESGGGVVQPGTSLRLSCAASQFRFDGYGMHWVRQAPGKGLEWVASISHDGIKKYHAEKVWGRFTISRDNSKNTLYLQMNSLRPEDTALYYCAKDLREDECEEWWSDYYDFGAQLPCAKSRGGLVGIADNWGQGTMVTVSS

SEQ ID NO: 112 - protein, 122 aa, synthetic construct
QSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTL

SEQ ID NO: 113 - protein, 60 aa, Haemaphysalis longicornis
YPERDSAKEGNQEQERALHVKVQKRTDGDADYDEYEEDGTTPTPDPTAPTAKPRLRGNKP

SEQ ID NO: 114 - protein, 70 aa, Haemaphysalis longicornis
QPKEKTKGVEVEGNPATLISARQMDVSYDEYEDNGPDVIPGEPAKPRGGPKNGAASGKFDQIPDFSSESH

SEQ ID NO: 115 - protein, 39 aa, Nipponia nippon
RHPFLEWSIQEPPAASYSGLELAEAMPSPRTIKTHLPVQ

SEQ ID NO: 116 - protein, 39 aa, Aptenodytes forsteri
RHPFLEWSVQESSAASYSGLELAEAMPSPRTIKTHLPVQ

SEQ ID NO: 117 - protein, 39 aa, Cariama cristata
RHPFLEWTMPEPPAMSYSGLELAEAMPSPRTIKTHLPAQ

SEQ ID NO: 118 - protein, 39 aa, Apaloderma vittatum
RHPFLEWFLPEPPSLRYSGLELAEAMPSPRTIKTHLPVQ

SEQ ID NO: 119 - protein, 39 aa, Opisthocomus hoazin
RHPFLEWSMEASPTLSYSGLELAEAMPFPRTIKTHLPVQ

SEQ ID NO: 120 - protein, 39 aa, Patagioenas fasciata
RHPFLEWYIPEPSSLRYSGLELAEAMPSPRTMKTHLPVQ

SEQ ID NO: 121 - protein, 39 aa, Gallus gallus
RHPFLEWYIPDSSPLGYSGLKLAEAMPSPRTMKTHLPVQ

SEQ ID NO: 122 - protein, 39 aa, Manacus vitellinus
RHPFLEWFVAEPPSERYSGVELANAMPSPRTIKIHLPVQ

SEQ ID NO: 123 - protein, 39 aa, Ficedula albicollis
RHPFLEWFIPEPPSMRYSGVELAEAMPSPRTIKTHLPVQ

SEQ ID NO: 124 - protein, 39 aa, Taeniopygia guttata
RHPFLEWYCAEPPSARYSGLELAEAMPSPRTMKTHLPVQ

SEQ ID NO: 125 - protein, 308 aa, Nipponia nippon
MALDKMEDLSLKETVVSRAEICEVEGIPFTKPICSTWDQVWKFKARPDDLLIATYTKAGTTWTQEIVDMIQQNGDVEKCRRATTYRRHPFLEWSIQEPPAASYSGLELAEAMPSPRTIKTHLPVQLLPPSFWEQNCKIIYVARNAKDNLVSYYHFHRMSKEMPDPGTWEEFMEKFMTGKVLWGSWYDHVKGWWKAKDRHRILYLFYEDMKENPKQEIQKILKFLEKDVNQEVLNKILHNTSFEIMKDNPMTNYTTEFQGIMDHSISPFMRKGVVGDWKNYFTVAQNEKFDEDYKKKMADTSLVFRTEL

SEQ ID NO: 126 - protein, 296 aa, Homo sapiens
MALTSDLGKQIKLKEVEGTLLQPATVDNWSQIQSFEAKPDDLLICTYPKAGTTWIQEIVDMIEQNGDVEKCQRAIIQHRHPFIEWARPPQPSGVEKAKAMPSPRILKTHLSTQLLPPSFWENNCKFLYVARNAKDCMVSYYHFQRMNHMLPDPGTWEEYFETFINGKVVWGSWFDHVKGWWEMKDRHQILFLFYEDIKRDPKHEIRKVMQFMGKKVDETVLDKIVQETSFEKMKENPMTNRSTVSKSILDQSISSFMRKGTVGDWKNHFTVAQNERFDEIYRRKMEGTSINFCMEL

SEQ ID NO: 127 - protein, 304 aa, Homo sapiens
MAKIEKNAPTMEKKPELFNIMEVDGVPTLILKSEWWEKVCNFQAKPDDLILATYPKSGTTWMHEILDMILNDGDVEKCKRAQTLDRHAFLELKFPHKEKPDLEFVLEMSSPQLIKTHLPSHLIPPSIWKENCKIVYVARNPKDCLVSYYHFHRMASFMPDPQNLEEFYEKFMSGKVVGGSWFDHVKGWWAAKDMHRILYLFYEDIKKDPKREIEKILKFLEKDISEEILNKIIYHTSFDVMKQNPMTNYTTLPTSIMDHSISPFMRKGMPGDWKNYFTVAQNEEFDKDYQKKMAGSTLTFRTEI

SEQ ID NO: 128 - protein, 296 aa, Homo sapiens
MLSPKDILRKDLKLVHGYPMICAFASNWEKIEQFHSRPDDIVIATYPKSGTTWVSEIIDMILNDGDIEKCKRGFITEKVPMLEMTLPGLRTSGIEQLEKNPSPRIVKTHLPTDLLPKSFWENNCKMIYLARNAKDVSVSYYHFDLMNNLQPFPGTWEEYLEKFLTGKVAYGSWFTHVKNWWKKKEEHPILFLYYEDMKENPKEEIKKIIRFLEKNLNDEILDRIIHHTSFEVMKDNPLVNYTHLPTTVMDHSKSPFMRKGTAGDWKNYFTVAQNEKFDAIYETEMSKTALQFRTEI

SEQ ID NO: 129 - protein, 295 aa, Homo sapiens
MELIQDTSRPPLEYVKGVPLIKYFAEALGPLQSFQARPDDLLISTYPKSGTTWVSQILDMIYQGGDLEKCHRAPIFMRVPFLEFKAPGIPSGMETLKDTPAPRLLKTHLPLALLPQTLLDQKVKVVYVARNAKDVAVSYYHFYHMAKVHPEPGTWDSFLEKFMVGEVSYGSWYQHVQEWWELSRTHPVLYLFYEDMKENPKREIQKILEFVGRSLPEETVDFVVQHTSFKEMKKNPMTNYTTVPQEFMDHSISPFMRKGMAGDWKTTFTVAQNERFDADYAEKMAGCSLSFRSEL

SEQ ID NO: 130 - protein, 295 aa, Homo sapiens
MELIQDISRPPLEYVKGVPLIKYFAEALGPLQSFQARPDDLLISTYPKSGTTWVSQILDMIYQGGDLEKCHRAPIFMRVPFLEFKVPGIPSGMETLKNTPAPRLLKTHLPLALLPQTLLDQKVKVVYVARNAKDVAVSYYHFYHMAKVYPHPGTWESFLEKFMAGEVSYGSWYQHVQEWWELSRTHPVLYLFYEDMKENPKREIQKILEFVGRSLPEETVDLMVEHTSFKEMKKNPMTNYTTVRREFMDHSISPFMRKGMAGDWKTTFTVAQNERFDADYAKKMAGCSLSFRSEL

SEQ ID NO: 131 - protein, 295 aa, Homo sapiens
MELIQDTSRPPLEYVKGVPLIKYFAEALGPLQSFQARPDDLLINTYPKSGTTWVSQILDMIYQGGDLEKCNRAPIYVRVPFLEVNDPGEPSGLETLKDTPPPRLIKSHLPLALLPQTLLDQKVKVVYVARNPKDVAVSYYHFHRMEKAHPEPGTWDSFLEKFMAGEVSYGSWYQHVQEWWELSRTHPVLYLFYEDMKENPKREIQKILEFVGRSLPEETMDFMVQHTSFKEMKKNPMTNYTTVPQELMDHSISPFMRKGMAGDWKTTFTVAQNERFDADYAEKMAGCSLSFRSEL

SEQ ID NO: 132 - protein, 294 aa, Homo sapiens
MNSELDYYEKFEEVHGILMYKDFVKYWDNVEAFQARPDDLVIATYPKSGTTWVSEIVYMIYKEGDVEKCKEDVIFNRIPFLECRKENLMNGVKQLDEMNSPRIVKTHLPPELLPASFWEKDCKIIYLCRNAKDVAVSFYYFFLMVAGHPNPGSFPEFVEKFMQGQVPYGSWYKHVKSWWEKGKSPRVLFLFYEDLKEDIRKEVIKLIHFLERKPSEELVDRIIHHTSFQEMKNNPSTNYTTLPDEIMNQKLSPFMRKGITGDWKNHFTVALNEKFDKHYEQQMKESTLKFRTEI

SEQ ID NO: 133 - protein, 285 aa, Homo sapiens
MSDDFLWFEGIAFPTMGFRSETLRKVRDEFVIRDEDVIILTYPKSGTNWLAEILCLMHSKGDAKWIQSVPIWERSPWVESEIGYTALSETESPRLFSSHLPIQLFPKSFFSSKAKVIYLMRNPRDVLVSGYFFWKNMKFIKKPKSWEEYFEWFCQGTVLYGSWFDHIHGWMPMREEKNFLLLSYEELKQDTGRTIEKICQFLGKTLEPEELNLILKNSSFQSMKENKMSNYSLLSVDYVVDKAQLLRKGVSGDWKNHFTVAQAEDFDKLFQEKMADLPRELFPWE

SEQ ID NO: 134 - protein, 365 aa, Homo sapiens
MDGPAEPQIPGLWDTYEDDISEISQKLPGEYFRYKGVPFPVGLYSLESISLAENTQDVRDDDIFIITYPKSGTTWMIEIICLILKEGDPSWIRSVPIWERAPWCETIVGAFSLPDQYSPRLMSSHLPIQIFTKAFFSSKAKVIYMGRNPRDVVVSLYHYSKIAGQLKDPGTPDQFLRDFLKGEVQFGSWFDHIKGWLRMKGKDNFLFITYEELQQDLQGSVERICGFLGRPLGKEALGSVVAHSTFSAMKANTMSNYTLLPPSLLDHRRGAFLRKGVCGDWKNHFTVAQSEAFDRAYRKQMRGMPTFPWDEDPEEDGSPDPEPSPEPEPKPSLEPNTSLEREPRPNSSPSPSPGQASETPHPRPS

SEQ ID NO: 135 - protein, 284 aa, Homo sapiens
MAESEAETPSTPGEFESKYFEFHGVRLPPFCRGKMEEIANFPVRPSDVWIVTYPKSGTSLLQEVVYLVSQGADPDEIGLMNIDEQLPVLEYPQPGLDIIKELTSPRLIKSHLPYRFLPSDLHNGDSKVIYMARNPKDLVVSYYQFHRSLRTMSYRGTFQEFCRRFMNDKLGYGSWFEHVQEFWEHRMDSNVLFLKYEDMHRDLVTMVEQLARFLGVSCDKAQLEALTEHCHQLVDQCCNAEALPVGRGRVGLWKDIFTVSMNEKFDLVYKQKMGKCDLTFDFYL

SEQ ID NO: 136 - protein, 303 aa, Homo sapiens
MADKSKFIEYIDEALEKSKETALSHLFFTYQGIPYPITMCTSETFQALDTFEARHDDIVLASYPKCGSNWILHIVSELIYAVSKKKYKYPEFPVLECGDSEKYQRMKGFPSPRILATHLHYDKLPGSIFENKAKILVIFRNPKDTAVSFLHFHNDVPDIPSYGSWDEFFRQFMKGQVSWGRYFDFAINWNKHLDGDNVKFILYEDLKENLAAGIKQIAEFLGFFLTGEQIQTISVQSTFQAMRAKSQDTHGAVGPFLFRKGEVGDWKNLFSEIQNQEMDEKFKECLAGTSLGAKLKYESYCQG

(19) United States
(12) Patent Application Publication
(10) Pub. No.: US 2024/0344040 A1
(43) Pub. Date: Oct. 17, 2024

(54) PROKARYOTIC AND EUKARYOTIC CELLS WITH BIOSYNTHESIZED SULFOTYROSINE FOR GENETIC INCORPORATION

(71) Applicant: William Marsh Rice University, Houston, TX (US)

(72) Inventors: Han XIAO, Houston, TX (US); Yuda CHEN, Houston, TX (US)

(73) Assignee: William Marsh Rice University, Houston, TX (US)

(21) Appl. No.: 18/613,852

(22) Filed: Mar. 22, 2024

Related U.S. Application Data
(60) Provisional application No. 63/492,168, filed on Mar. 24, 2023.

(51) Int. Cl. C12N 9/10; A61K 38/00; C07K 14/81; C12N 9/00

(52) U.S. Cl. CPC C12N 9/13; C07K 14/811; C12N 9/93; C12Y 208/02001; C12Y 601/01001; A61K 38/00; C12N 2510/02

(57) ABSTRACT

The present disclosure provides an engineered cell comprising a sulfotransferase. In some embodiments, the sulfotransferase is NnSULT1C1 sulfotransferase. The present disclosure also provides methods for the biosynthesis of a peptide containing at least one sulfotyrosine residue, as well as compositions comprising a peptide containing at least one sulfotyrosine residue. In some embodiments, the compositions and methods of the present disclosure provide therapeutic peptides for use in treating and/or preventing a disease or disorder, such as an HIV-1 infection.

### PRIORITY CLAIM

This application claims benefit of priority to U.S. Provisional Application Ser. No. 63/492,168, filed Mar. 24, 2023, the entire contents of which are hereby incorporated by reference.

### STATEMENT REGARDING FEDERALLY SPONSORED RESEARCH

This invention was made with government support under Grant No. R35-GM133706 awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said XML Sequence Listing, created on Mar. 21, 2024, is named RICEP0129US.xml and is 127,734 bytes in size.

### BACKGROUND

### 1. Field

The present disclosure relates generally to the fields of molecular biology and biosynthesis. Methods and compositions that use sulfotransferases, such as NnSULT1C1 sulfotransferase are provided. Sulfotransferases described herein may be used for the synthesis of sulfotyrosine, which can be incorporated into peptides to enhance therapeutic efficiency.

### 2. Description of Related Art

With the rare exceptions of pyrrolysine and selenocysteine, a standard set of 20 amino acid building blocks, containing a limited number of functional groups, is used by almost all organisms for the biosynthesis of proteins. The use of Genetic Code Expansion technology to enable the site-specific incorporation of noncanonical amino acids (ncAAs) into proteins in living cells has transformed our ability to study biological processes and develop modern medicines (Wang et al., 2006; Ambrogelly et al., 2007; Liu & Schultz, 2010; Chin, 2014; Dien et al., 2018; Chin, 2017). The genetic encoding of ncAAs with distinct chemical, biological, and physical properties requires the engineering of bioorthogonal translational machinery, consisting of an evolved aminoacyl-tRNA synthetase/tRNA pair and a “blank” codon (Wang et al., 2006; Chin, 2017; Wang et al., 2001). The high intracellular concentration of ncAA required to render this machinery operative has usually been achieved via chemical synthesis of the ncAA and its exogenous addition at high levels to the cell culture medium. However, unlike canonical amino acids, ncAAs lack specific and efficient transporters that enable cellular internalization. Furthermore, lipid bilayers are impermeable to most polar and charged ncAAs (Luo et al., 2017; Hoppmann et al., 2017; Burkovski & Krämer, 2002; Palacin et al., 1998; Bundy & Swartz, 2010). These factors greatly limit the efficiency of ncAA incorporation into proteins using the Genetic Code Expansion technology (Luo et al., 2017; Burkovski & Krämer, 2002; Palacin et al., 1998; Bundy & Swartz, 2010; Zhang et al., 2017; Ko et al., 2019).

Strategies for engineering the structures of ncAAs or ncAA-binding proteins have been employed to improve the cellular uptake of ncAAs. In 2017, the Schultz group adopted a dipeptide strategy to enable the cellular uptake of phosphotyrosine. The phosphotyrosine-containing dipeptide can be synthesized and transported into cells via an adenosine triphosphate (ATP)-binding cassette transporter, followed by hydrolysis of the dipeptide by nonspecific intracellular peptidases (Luo et al., 2017). In the same year, the Wang lab developed a two-step strategy for producing proteins with site-specific tyrosine phosphorylation (Hoppmann et al., 2017). This strategy utilized the incorporation of a phosphotyrosine analogue with a cage group, followed by chemical deprotection of the purified proteins. But the synthesis and purification of these dipeptides are challenging, and the required post-purification treatments limit the applicability of this methodology to efficient incorporation of phosphotyrosine into proteins. As an alternative approach, periplasmic binding proteins (PBPs) have been engineered to have improved affinities for specific ncAAs (Ko et al., 2019). These mutant PBPs enhanced uptake of the respective ncAAs up to 5-fold, as evidenced by elevated intracellular ncAA concentrations and the yield of ncAA-containing green fluorescent proteins (Ko et al., 2019). Nevertheless, the engineered PBP species are only applicable to a subset of ncAAs, and exogenous feeding of high concentrations of the ncAAs is still required. The problem of ncAA uptake could potentially be bypassed by intracellular biosynthesis of the ncAAs (Zhang et al., 2017; Chen et al., 2018; Rogerson et al., 2015; Chen et al., 2020). For example, phosphothreonine (pThr) cannot be detected intracellularly even when cells are incubated with 1 mM pThr (Zhang et al., 2017). The Chin group overcame the membrane impermeability of pThr by introducing the Salmonella enterica kinase, PduX, which converts L-threonine to pThr intracellularly (Zhang et al., 2017). This biosynthesis of pThr generated intracellular pThr at levels greater than 1 mM, sufficient for genetic incorporation of this amino acid (Zhang et al., 2017). A similar strategy was recently applied to the creation of autonomous bacterial cells that can biosynthesize and genetically incorporate p-amino-phenylalanine (pAF), 5-hydroxyl-tryptophan (5HTP) and dihydroxyphenylalanine (DOPA), although no autonomous eukaryotic cells have been reported (Chen et al., 2018; Chen et al., 2020; Chen et al., 2021). Additional biosynthetic pathways for producing polar or negatively-charged ncAAs would greatly expand the utility of genetic code expansion methods.

Tyrosine sulfation is an important post-translational modification of proteins that is important for a variety of biomolecular interactions, including chemotaxis, viral infection, anti-coagulation, cell adhesion, and plant immunity (Veldkamp et al., 2008; Ludeman & Stone, 2014; Farzan et al., 1999; Choe et al., 2003; Thompson et al., 2017; Somers et al., 2000; Westmuckett et al., 2011; Lee et al., 2009). For example, tyrosine sulfation of hirudin increases its affinity for thrombin more than 10-fold (Hsieh et al., 2014; Corral-Rodriguez et al., 2010). Thrombin inhibitors represent an important class of anticoagulants used to prevent blood clotting. In addition, several thrombin inhibitors from hematophagous organisms have been shown to facilitate the acquisition and digestion of bloodmeal (Tanaka-Azevedo et al., 2010; Koh & Kini; Kazimirová et al., 2013). Recent studies have reported that post-translational sulfation of these proteins has a dramatic effect on their inhibitory activity (Hsieh et al., 2014; Thompson et al., 2017).

Despite its importance and ubiquity, protein sulfation has been difficult to study due to the lack of general methods for preparing proteins with defined sulfated residues (Thompson et al., 2017; Li et al., 2018). To circumvent this challenge, efforts have been previously made to site-specifically incorporate sulfotyrosine using the Genetic Code Expansion technology (Liu & Schultz, 2006). The resulting sulfotyrosine incorporation systems have enabled several applications, including generation of therapeutic proteins with defined sulfated tyrosines, evolution of sulfated anti-gp120 antibodies, and confirmation of tyrosine sulfation sites (Li et al., 2018; Liu et al., 2009; Liu et al., 2008; Liu et al., 2009b; Schwessinger et al., 2016). To achieve reasonable expression levels of sulfated proteins in E. coli, however, most studies have required the exogenous feeding of 3-20 mM sulfotyrosine to compensate for low intracellular uptake of extracellular sulfotyrosine (Liu et al., 2009; Liu et al., 2008; Liu et al., 2009b; Schwessinger et al., 2016). Thus, a need exists for alternate methods of sulfotyrosine biosynthesis.

### SUMMARY OF THE INVENTION

Provided herein are methods and compositions that use sulfotransferases, such as NnSULT1C1 sulfotransferase. In some aspects, the present disclosure provides an engineered cell comprising a sulfotransferase having a sequence that is at least about 95% identical to the amino acid sequence of SEQ ID NO: 1. In some embodiments, the sulfotransferase has a sequence that is identical to the amino acid sequence of SEQ ID NO: 1:

(SEQ ID NO: 1)
IALDKMEDLSLKETVVSRAEICEVEGIPFTKPICSTWDQVWKFKARPDDL
LIATYTKAGTTWTQEIVDMIQQNGDVEKCRRATTYRRHPFLEWSIQEPPA
ASYSGLELAEAMPSPRTIKTHLPVQLLPPSFWEQNCKIIYVARNAKDNLV
SYYHFHRMSKEMPDPGTWEEFMEKFMTGKVLWGSWYDHVKGWWKAKDRHR
ILYLFYEDMKENPKQEIQKILKFLEKDVNQEVLNKILHNTSFEIMKDNPM
TNYTTEFQGIMDHSISPFMRKGVVGDWKNYFTVAQNEKFDEDYKKKMADT
SLVFRTEL

In some embodiments, the sulfotransferase is a tyrosine sulfotransferase. In certain embodiments, the engineered cell further comprises a tyrosyl-tRNA synthetase/tRNA pair. In some embodiments, the tyrosyl-tRNA synthetase/tRNA pair is derived from E. coli. In some embodiments, the engineered cell further comprises 3′-phosphoadenosine-5′-phosphosulfate. In some embodiments, the engineered cell further comprises a peptide comprising sulfotyrosine at one or more positions. In certain embodiments, the peptide comprises sulfotyrosine at two or more positions.

In some embodiments, the peptide has a sequence that is at least about 90% identical to SEQ ID NO: 107. In certain embodiments, the peptide has a sequence that is at least about 95% identical to SEQ ID NO: 107. In some embodiments, the peptide has a sequence that is at least about 90% identical to SEQ ID NO: 108. In certain embodiments, the peptide has a sequence that is at least about 95% identical to SEQ ID NO: 108. In some embodiments, the peptide has a sequence that is at least about 90% identical to SEQ ID NO: 109. In certain embodiments, the peptide has a sequence that is at least about 95% identical to SEQ ID NO: 109. In some embodiments, the peptide has a sequence that is at least about 90% identical to SEQ ID NO: 110. In certain embodiments, the peptide has a sequence that is at least about 95% identical to SEQ ID NO: 110. In some embodiments, the peptide has a sequence that is at least about 90% identical to SEQ ID NO: 111. In certain embodiments, the peptide has a sequence that is at least about 95% identical to SEQ ID NO: 111. In some embodiments, the peptide has a sequence that is at least about 90% identical to SEQ ID NO: 112. In certain embodiments, the peptide has a sequence that is at least about 95% identical to SEQ ID NO: 112.

In some embodiments, the peptide is a thrombin-inhibitor. In some embodiments, the engineered cell is a mammalian cell or a prokaryotic cell. In certain embodiments, the engineered cell is a prokaryotic cell, such as an E. coli cell. In some embodiments, the cell further expresses ATP sulfurylase, adenosine 5′-phosphosulfate kinase, and adenosine-3′,5′-diphosphate nucleotidase.

In some embodiments, the engineered cell is a eukaryotic cell. In certain embodiments, the engineered cell is a human embryonic kidney (HEK) cell, such as a HEK293T cell. In some embodiments, the cellular concentration of sulfotyrosine is greater than or equal to 500 PM. In certain embodiments, the cellular concentration of sulfotyrosine is greater than or equal to 700 [M. In some embodiments, the cellular concentration of sulfotyrosine is about 750 PM.

In some aspects, the present disclosure provides a method of expressing a recombinant peptide comprising at least one sulfotyrosine residue at a selected position not found in a wild-type version of the peptide, the method comprising:
a. obtaining an engineered cell that comprises 3′-phosphoadenosine-5′-phosphosulfate and a sulfotransferase that is at least about 95% identical to SEQ ID NO: 1;
b. expressing a nucleic acid encoding the recombinant peptide in the cell; and
c. purifying the recombinant peptide from the cell.

In some embodiments, the cell comprises an expression construct encoding the sulfotransferase operably linked to an arabinose-inducible promoter, wherein the method further comprises inducing sulfotransferase expression with L-arabinose. In some embodiments, L-arabinose is present at a concentration of 15 mg/mL. In some embodiments, the method produces at least about 4 mg/L of sulfotyrosine. In certain embodiments, the method produces at least about 5 mg/L of sulfotyrosine. In some embodiments, the cellular concentration of sulfotyrosine is greater than or equal to 500 μM. In further embodiments, the cellular concentration of sulfotyrosine is greater than or equal to 700 μM. In still further embodiments, the cellular concentration of sulfotyrosine is about 750 μM.

In some aspects, the present disclosure describes a composition comprising the purified recombinant peptide produced by a method described herein. In some embodiments, at least 80% of the recombinant peptides in the composition comprise the sulfotyrosine residue at the selected position.

In some embodiments, the recombinant peptide has a sequence that is at least about 90% identical to SEQ ID NO: 107. In certain embodiments, the recombinant peptide has a sequence that is at least about 90% identical to SEQ ID NO: 108. In some embodiments, the recombinant peptide has a sequence that is at least about 90% identical to SEQ ID NO: 109. In other embodiments, the recombinant peptide has a sequence that is at least about 90% identical to SEQ ID NO: 110. In other embodiments, the recombinant peptide has a sequence that is at least about 90% identical to SEQ ID NO: 111. In yet other embodiments, the recombinant peptide has a sequence that is at least about 90% identical to SEQ ID NO: 112. In some embodiments, the recombinant peptide is a thrombin-inhibitor.

In some aspects, the present disclosure provides a pharmaceutical composition comprising a composition described herein. In some aspects, the present disclosure provides a method of treating a subject in need thereof, the method comprising administering an effective amount of a pharmaceutical composition described herein to the subject. In some embodiments, the pharmaceutical composition may be used in the treatment or prevention of a disease or disorder. In some embodiments, the composition may be used for treating or preventing blood clots. In some embodiments, the composition may be used for treating or preventing an HIV-1 infection.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

The patent or application file contains at least one drawing executed in color. Copies of this patent or patent application publication with color drawing(s) will be provided by the Office upon request and payment of the necessary fee.

FIGS. 1A-D: Discovery of tyrosine sulfotransferase from sequence similar network: (A) sulfotyrosine was biosynthesized from tyrosine and PAPS in the presence of sulfotransferase identified in this study. The resulting biosynthesized sulfotyrosine was site-specifically incorporated into thrombin inhibitors, yielding enhanced thrombin inhibition (B) Sequence similar network (SSN) generated by EFI-EST server with RnSULT1A1 as an input sequence and E-value of 5. Each circle stands for a representative node containing sequences with over 80% identity. Edges detection threshold was set at an alignment score of 110. The upper and lower representative nodes are RnSULT1A1 (P17988) and HsSULT1C2 (000338), respectively. (C) Schematic representation of reported sulfation reactions of P17988 and 000338. (D) Screening of tyrosine sulfotransferases with green fluorescent protein assay. All tested proteins are included in the representative nodes of (B) and NnSULT1C1 is the protein with red label (B).

FIGS. 2A-J: Exploring the mechanism of unique tyrosine specificity of NnSULT1C1: (A) NnSULT1C1 structure predicted by AlphaFold2 and its active site consisting of PAPS and Tyr. Tyr was docked into NnSULT1C1 containing PAPS by Glide v8.1 in Schrödinger software. (B) Green fluorescent protein assay with wildtype NnSULT1C1 (wt) or NnSULT1C1 without the SIQEPPAASY (Δloop). (C) Green fluorescent protein assay with wildtype NnSULT1C1 (wt) or NnSULT1C1 with alanine mutation at indicated residues. (D) Structural similarity search of NnSULT1C1 using the PDBeFold web server. (E) Characterization of Tyr docking with NnSULT1C1 and its structurally similar sulfotransferases via docking score and nucleophilic attack distance. Docking scores were calculated using Glide v8.1 in Schrödinger software. Nucleophilic attack distance was defined as the distances between Tyr phenolic alcohol and PAPS sulfonate. (F) Comparison of tyrosine sulfation activity of NnSULT1C1 and its structurally similar sulfotransferases using green fluorescent protein assay. Cells without any sulfotransferase (−) were used as control. (G-J) Tyr docking position with NnSULT1C1 (G), mSULT1D1 (H), hSULT1A3 (I), and hSULT1C2 (J). PAPS and Tyr are shown as sticks with carbon. Docking was performed by Glide v8.1 in Schrödinger software with the same parameters in (A). *p<0.05; ****p<0.0001.

FIGS. 3A-H: Generation of completely autonomous sulfotyrosine synthesizing E. coli: (A) Schematic representation of genetic circuits used for generating completely autonomous sulfotyrosine synthesizing E. coli. (B) Screening of the knockout strains for sfGFP-sulfotyrosine production after the expression of NnSULT1C1. (C) The roles of PAPS recycling enzymes in producing sfGFP-sulfotyrosine using ΔcysH BW25113 strain. (D) Cellular concentrations of sulfotyrosine of cells with the addition of chemically synthesized sulfotyrosine or the biosynthesis of sulfotyrosine. (E) Production of sfGFP-sulfotyrosine from cells with the addition of chemically synthesized sulfotyrosine or the biosynthesized Tyr. The effect of NnSULT1C1 expression level on producing sfGFP-sulfotyrosine was screened by altering the concentration of inducer, l-arabinose (l-ara). (F) SDS-PAGE analysis of sfGFPs expressed in LB in the presence (+) or absence (−) of exogenous 1 mM sulfotyrosine addition or when inducing NnSULT1C1 expression (bio). (G-H) Mass spectra analysis of sfGFP-sulfotyrosine proteins expressed in cells with the addition of 1 mM chemically synthesized sulfotyrosine or the biosynthesis of sulfotyrosine.

FIGS. 4A-D: Generation of completely autonomous mammalian cells with sulfotyrosine-containing proteins: (A) Schematic representation of genetic circuits used for generating completely autonomous mammalian cells with sulfotyrosine-containing proteins. (B) Confocal images of HEK293T (exogenously fed) and HEK293T-NnSULT1C1 (bio) cells expressing sulfotyrosineRS, tRNA_{CUA} and EGFP containing an amber codon at Tyr39 position. Scale bar=μm. (C) Flow cytometric analysis of EGFP expression levels of HEK293T (exogenously fed) and HEK293T-NnSULT1C1 (bio) cells with sulfotyrosineRS, tRNA_{CUA} and EGFP containing an amber codon at Tyr39 position. The normalized fluorescence was calculated by multiplying the geometric mean fluorescence by the percentage of EGFP-positive cells. Error bars represent standard deviations. (D) Mass spectra analysis of EGFP with sulfotyrosine (EGFP-39-sulfotyrosine) purified from HEK293T-NnSULT1C1 cells.

FIGS. 5A-E: Production of thrombin inhibitors with site-specific sulfotyrosine insertion using completely autonomous E. coli: (A) Madanin-1 with sulfation at Tyr32 and Tyr35 positions binds to exosite II site in thrombin. (B) Amino acid sequences of madanin-1 and chimadanin (SEQ ID NOs: 113 and 114) (sulfation sites are shown). (C) SDS-PAGE analysis of thrombin inhibitors with site-specific sulfotyrosine insertion expressed in completely autonomous E. coli. (D-E) Inhibition of thrombin activity by madanin-1 and chimadanin proteins. Error bars represent standard deviation.

FIG. 6: Screening reported sulfotransferases with GFP assay: Data are plotted as means from n=2 independent samples. a.u. stands for arbitrary unit.

FIG. 7: Phylogenetic relationship of all sulfotransferases tested in FIG. 2d: Phylogenetic tree was generated in MEGAX software with UPGMA method. A0A091VQH7 was named NnSULT1C1 and used for following experiments.

FIG. 8: Phylogenetic analysis and multiple sequence alignment of NnSULT1C1 and its 9 relatives: Phylogenetic tree was constructed by UPGMA method in MEGAX and multiple sequences (SEQ ID NOs: 115-124) were aligned by ClustalW method.

FIG. 9: Sequence alignment of human cytosolic sulfotransferases (hSULTs) and NnSULT1C1: The highly variable region (SIQEPPAAS) in NnSULT1C1 is aligned well with the reported residues of hSULTs important for substrate recognition (SEQ ID NOs: 125-136).

FIG. 10: Superimposition of NnSULT1C1 and 2zvq: NnSULT1C1 and mouse SULT1D1 (PDB: 2zvq) are shown. The variable region SIQEPPAAS of NnSULT1C1 is also shown.

FIG. 11: Expression condition screening for sfGFP-sulfotyrosine production: The influence of expression medium, tyrosine addition, sulfate addition and glycerol addition on production of sfGFP-sulfotyrosine in bacterial cells containing sulfotyrosine biosyntheis and genetic incorporation machineries was evaluated with green fluorescent protein assay. Data are plotted as the mean+/−standard deviation from n=3 independent samples. a.u. stands for arbitrary unit.

FIGS. 12A-D: Kinetics measurement of tyrosine sulfation activity of NnSULT1C1: (A) SDS-PAGE analysis of NnSULT1C1-His6 expressed in LB medium. (B) Standard curve of authentic sulfotyrosine detected by SIM mode of ESI-MS. Data are plotted as means of n=2 independent samples. (C) The effect of adding his6 tag to C terminal of NnSULT1C1 on its activity. (D) Kinetics curve of NnSULT1C1 with tyrosine as its substrate. Data are plotted as means of n=3 independent samples. Error bars represent standard deviations from n=3 independent samples. Vₘₐₓ and Kₘ were obtained by fitting the data to Michaelis-Menten equation in Prism. a.u. stands for arbitrary unit.

FIG. 13: Cellular concentration of sulfotyrosine in HEK293T and HEK293T-NnSULT1C1: Indicated concentration of sulfotyrosine was added to the culture of HEK293T or HEK293T-NnSULT1C1 for 2 hours. Data are plotted as means from n=2 independent groups.

FIG. 14: ESI-MS analysis of EGFP39sulfotyrosine from HEK293T cells and HEK293T-NnSULT1C1. The expected peak was calculated according to monoisotopic mass of EGFP39sY with N-terminal acetylation. Bottom left spectrum is identical to FIG. 4D. a.u. stands for arbitrary unit.

FIG. 15: SDS-PAGE analysis of thrombin inhibitors purified from LB medium: sulfotyrosine-containing inhibitors are expressed in LB medium with external addition of 3 mM sulfotyrosine.

FIG. 16: Protein yields of all thrombin inhibitors used in this study.

FIG. 17: ESI-MS analysis of all thrombin inhibitors used in this study. a.u. stands for arbitrary unit.

FIG. 18: Inhibition constants (Ki) of all thrombin inhibitors used in this study: Kᵢ+standard error were calculated based on a tight-binding model, using Morrison equation in Prism. Standard errors were calculated from n=3 independent samples.

FIG. 19: Original gel for FIG. 3F.

FIG. 20: Original gel for Madanin-1. (FIG. 3F left and FIG. 16 left)

FIG. 21: Original gel for Chimadanin. (FIG. 3F right and FIG. 16 right)

FIG. 22: Original gel for FIG. 12 (The lane next to ladder is NnSULT1C1).

### DETAILED DESCRIPTION

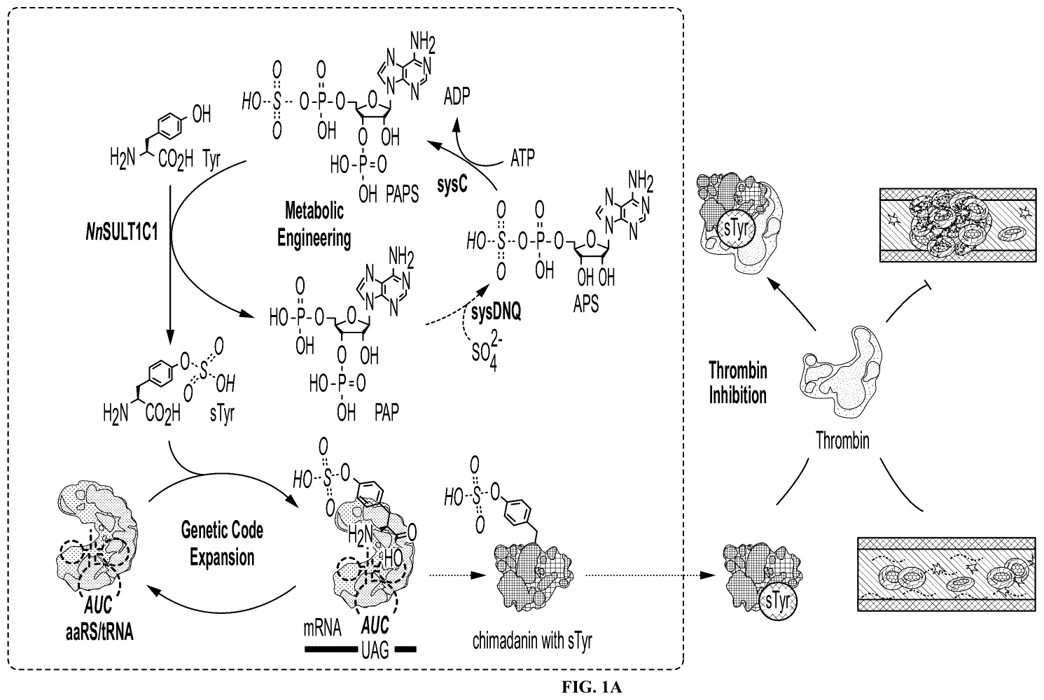

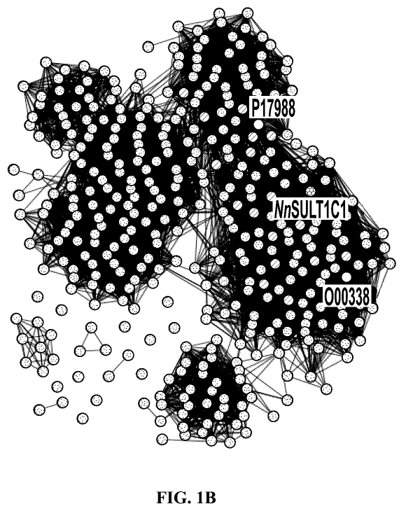

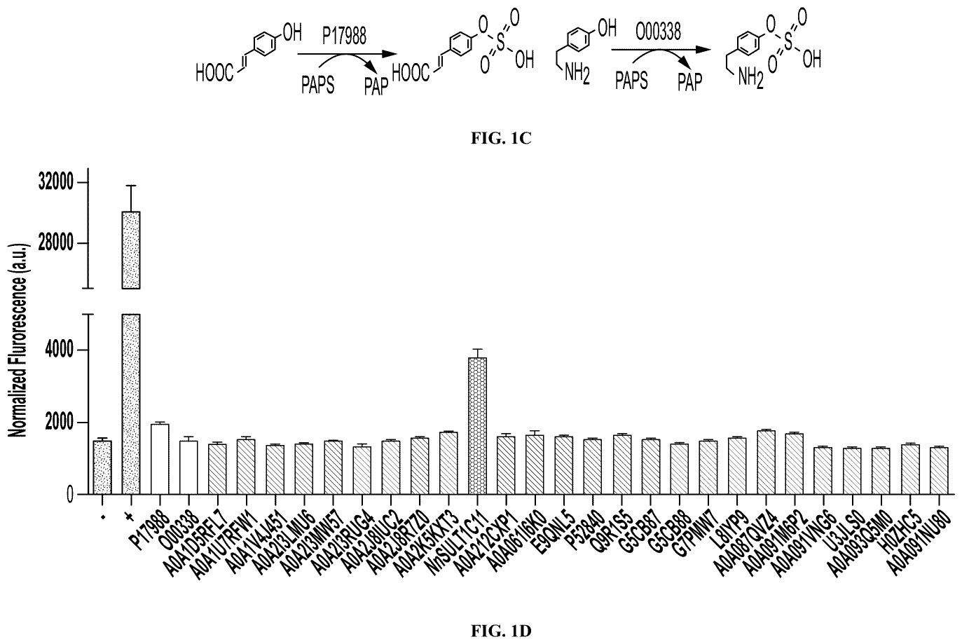

Described herein is the generation of completely autonomous prokaryotic and eukaryotic organisms capable of incorporating sulfotyrosine into proteins (e.g., FIG. 1A). Sulfotyrosine is biosynthesized using a new sulfotransferase discovered using a sequence similarity network (SSN). Sulfotyrosine is subsequently incorporated into proteins in response to a repurposed stop codon. The molecular properties of this new sulfotransferase were explored using bioinformatics and computational approaches, revealing a loop structure and several residues in binding pocket within this enzyme responsible for its unique specificity for tyrosine. The further optimization of the genome and sulfotyrosine biosynthetic pathway of both prokaryotic and eukaryotic cells leads to greater expression yields of sulfated proteins than experienced with cells exogenously fed with sulfotyrosine. The utility of these autonomous cells is demonstrated by using them to produce highly potent thrombin inhibitors.

These and other aspects of the disclosure are described in detail below.

### I. TYROSINE SULFATION

Sulfotransferases are transferase enzymes that catalyze the transfer of a sulfo group (R—SO₃) from a donor molecule to an acceptor alcohol (R—OH) or amine (R—NH₂) (Negishi et al., 2001) The most common sulfo group donor is 3′-phosphoadenosine-5′-phosphosulfate (PAPS). In the case of alcohol as acceptor, the product is a sulfate (R—OSO₃): whereas an amine leads to a sulfamate (R—NH—SO₃): Both reactive groups for a sulfonation via sulfotransferases may be part of a protein, lipid, carbohydrate or steroid (Rath et al., 2004). In nature, sulfotransferases allow many organisms to utilize PAPS, for biosynthetic purposes (Yang et al., 2015; Gamage et al., 2006).

Based on their substrate preference and cellular location, sulfotransferases can be grouped into three major families, tyrosylprotein sulfotransferase (TPST), cytosolic sulfotransferase (SULT), and carbohydrate sulfotransferase (CHST) (Allali-Hassani et al., 2007; Suiko et al., 2017). Cytosolic sulfotransferases (SULTs) catalyze sulfation of a wide variety of endogenous compounds, including hormones, neurotransmitters, and xenobiotics (Allali-Hassani et al., 2007).

SULTS may also catalyze the sulfation of steroids and catecolamines (Suiko et al., 2017). Post-translational tyrosine sulfation occurs exclusively in eukaryotes. Although this modification has been estimated to occur on 1% of all tyrosine residues in eukaryotic proteomes, its functional significance is not well understood (Yang et al., 2015; Moore, 2009; Seibert & Sakmar, 2008). One approach to determining the biological importance of protein tyrosine sulfation is to express in living cells proteins that are sulfated in site-specific and homogeneous fashion, a goal that is difficult to achieve by chemical synthesis or recombinant expression. Genetic code expansion based on E. coli-derived tyrosyl-tRNA synthetase (EcTyrRS)/tRNA has been proven to overcome these challenges by site-specifically incorporating sulfotyrosine in proteins in mammalian cells (He et al., 2020; Italia et al., 2020).

### II. PEPTIDES OF INTEREST

In some aspects, the present disclosure provides compositions and methods for the use of sulfotransferases, such as a sulfotransferase of SEQ ID NO: 1 or a variant thereof to create sulfotyrosine. In some embodiments, sulfotyrosine may be incorporated into a peptide of interest. In some embodiments, the average molecular weight of the peptide of interest is from about 0.5 kDa to about 500 kDa. In some embodiments, the average molecular weight of the peptide of interest is from about 2.5 kDa to about 175 kDa. In some embodiments, the average molecular weight of the peptide of interest is from about 5 kDa about 150 kDa. In some embodiments, the average molecular weight of the peptide of interest is from about 10 kDa to about 125 kDa. In some embodiments, the average molecular weight of the peptide of interest is from about 12.5 kDa to about 100 kDa. In some embodiments, the average molecular weight of the peptide of interest is from about 15 kDa to about 90 kDa. In some embodiments, the average molecular weight of the peptide of interest is from about 17.5 kDa to about about 80 kDa. In some embodiments, the average molecular weight of the peptide of interest is from about 20 kDa to about 70 kDa. In some embodiments, the average molecular weight of the peptide of interest is from about 22.5 kDa to about 60 kDa. In some embodiments, the average molecular weight of the peptide of interest is from about 25 kDa to about 50 kDa.

In some embodiments, the peptide of interest may comprise at least 0.001% sulfotyrosine. In some embodiments, the peptide of interest may comprise at least 0.01% sulfotyrosine. In some embodiments, the peptide of interest may comprise at least 0.025% sulfotyrosine. In some embodiments, the peptide of interest may comprise at least 0.05% sulfotyrosine. In some embodiments, the peptide of interest may comprise at least 0.075% sulfotyrosine. In some embodiments, the peptide of interest may comprise at least 0.1% sulfotyrosine. In some embodiments, the peptide of interest may comprise at least 0.25% sulfotyrosine. In some embodiments, the peptide of interest may comprise at least 0.5% sulfotyrosine. In some embodiments, the peptide of interest may comprise at least 0.75% sulfotyrosine. In some embodiments, the peptide of interest may comprise at least 1.0% sulfotyrosine. In some embodiments, the peptide of interest may comprise at least 1.5% sulfotyrosine. In some embodiments, the peptide of interest may comprise at least 2.0% sulfotyrosine. In some embodiments, the peptide of interest may comprise at least 3.0% sulfotyrosine. In some embodiments, the peptide of interest may comprise at least 4.0% sulfotyrosine. In some embodiments, the peptide of interest may comprise at least 5.0% sulfotyrosine. In some embodiments, the peptide of interest may comprise at least 10.0% sulfotyrosine.

In some embodiments, the peptide of interest comprises a sequence that is at least 50% identical to SEQ ID NO: 107. In some embodiments, the peptide of interest comprises a sequence that is at least 60% identical to SEQ ID NO: 107. In some embodiments, the peptide of interest comprises a sequence that is at least 70% identical to SEQ ID NO: 107. In some embodiments, the peptide of interest comprises a sequence that is at least 80% identical to SEQ ID NO: 107. In some embodiments, the peptide of interest comprises a sequence that is at least 90% identical to SEQ ID NO: 107. In some embodiments, the peptide of interest comprises a sequence that is at least 95% identical to SEQ ID NO: 107. In some embodiments, the peptide of interest comprises a sequence that is at least 99% identical to SEQ ID NO: 107.

In some embodiments, the peptide of interest comprises a sequence that is at least 50% identical to SEQ ID NO: 108. In some embodiments, the peptide of interest comprises a sequence that is at least 60% identical to SEQ ID NO: 108. In some embodiments, the peptide of interest comprises a sequence that is at least 70% identical to SEQ ID NO: 108. In some embodiments, the peptide of interest comprises a sequence that is at least 80% identical to SEQ ID NO: 108. In some embodiments, the peptide of interest comprises a sequence that is at least 90% identical to SEQ ID NO: 108. In some embodiments, the peptide of interest comprises a sequence that is at least 95% identical to SEQ ID NO: 108. In some embodiments, the peptide of interest comprises a sequence that is at least 99% identical to SEQ ID NO: 108.

In some embodiments, the peptide of interest comprises a sequence that is at least 50% identical to SEQ ID NO: 109. In some embodiments, the peptide of interest comprises a sequence that is at least 60% identical to SEQ ID NO: 109. In some embodiments, the peptide of interest comprises a sequence that is at least 70% identical to SEQ ID NO: 109. In some embodiments, the peptide of interest comprises a sequence that is at least 80% identical to SEQ ID NO: 109. In some embodiments, the peptide of interest comprises a sequence that is at least 90% identical to SEQ ID NO: 109. In some embodiments, the peptide of interest comprises a sequence that is at least 95% identical to SEQ ID NO: 109. In some embodiments, the peptide of interest comprises a sequence that is at least 99% identical to SEQ ID NO: 109.

In some embodiments, the peptide of interest comprises a sequence that is at least 50% identical to SEQ ID NO: 110. In some embodiments, the peptide of interest comprises a sequence that is at least 60% identical to SEQ ID NO: 110. In some embodiments, the peptide of interest comprises a sequence that is at least 70% identical to SEQ ID NO: 110. In some embodiments, the peptide of interest comprises a sequence that is at least 80% identical to SEQ ID NO: 110. In some embodiments, the peptide of interest comprises a sequence that is at least 90% identical to SEQ ID NO: 110. In some embodiments, the peptide of interest comprises a sequence that is at least 95% identical to SEQ ID NO: 110. In some embodiments, the peptide of interest comprises a sequence that is at least 99% identical to SEQ ID NO: 110.

In some embodiments, the peptide of interest comprises a sequence that is at least 50% identical to SEQ ID NO: 111. In some embodiments, the peptide of interest comprises a sequence that is at least 60% identical to SEQ ID NO: 111. In some embodiments, the peptide of interest comprises a sequence that is at least 70% identical to SEQ ID NO: 111. In some embodiments, the peptide of interest comprises a sequence that is at least 80% identical to SEQ ID NO: 111. In some embodiments, the peptide of interest comprises a sequence that is at least 90% identical to SEQ ID NO: 111. In some embodiments, the peptide of interest comprises a sequence that is at least 95% identical to SEQ ID NO: 111. In some embodiments, the peptide of interest comprises a sequence that is at least 99% identical to SEQ ID NO: 111.

In some embodiments, the peptide of interest comprises a sequence that is at least 50% identical to SEQ ID NO: 112. In some embodiments, the peptide of interest comprises a sequence that is at least 60% identical to SEQ ID NO: 112. In some embodiments, the peptide of interest comprises a sequence that is at least 70% identical to SEQ ID NO: 112. In some embodiments, the peptide of interest comprises a sequence that is at least 80% identical to SEQ ID NO: 112. In some embodiments, the peptide of interest comprises a sequence that is at least 90% identical to SEQ ID NO: 112. In some embodiments, the peptide of interest comprises a sequence that is at least 95% identical to SEQ ID NO: 112. In some embodiments, the peptide of interest comprises a sequence that is at least 99% identical to SEQ ID NO: 112.

In some embodiments, the peptide of interest comprises a sequence that is at least 50% identical to a sequence listed in Table 1. In some embodiments, the peptide of interest comprises a sequence that is at least 60% identical to a sequence listed in Table 1. In some embodiments, the peptide of interest comprises a sequence that is at least 70% identical to a sequence listed in Table 1. In some embodiments, the peptide of interest comprises a sequence that is at least 80% identical to a sequence listed in Table 1. In some embodiments, the peptide of interest comprises a sequence that is at least 90% identical to a sequence listed in Table 1. In some embodiments, the peptide of interest comprises a sequence that is at least 95% identical to a sequence listed in Table 1. In some embodiments, the peptide of interest comprises a sequence that is at least 99% identical to a sequence listed in Table 1.

TABLE 1
Peptides of Interest

| | | SEQ |
|---|---|---|
| | | ID |
| Name | Sequence | NO |
| Madanin-1 | MQPKEKTKGVEVEGNPATLISARQMDVSYDEY | 107 |
| | EDNGPDVIPGEPAKPRGGPKNGAASGKFDQIP | |
| | DFSSESHHHHHHH | |
| Chimadanin | MYPERDSAKEGNQEQERALHVKVQKRTDGDAD | 108 |
| | YDEYEEDGTTPTPDPTAPTAKPRLRGNKPHHH | |
| | HHH | |
| Hirudin | MKKNIAFLLASMFVFSIATNAYAMRYTACTES | 109 |
| | GQNQCICEGNDVCGQGRNCQFDSSGKKCVEGE | |
| | GTRKPQNEGQHDFDPIPEEYLSHHHHHH | |
| V2 peptide | KVQKEYALFYELDIVPID | 110 |
| of HIV-1 | | |
| gp120 | | |
| CAP256V2LS: | QVQLVESGGGVVQPGTSLRLSCAASQFRFDGY | 111 |
| Variable | GMHWVRQAPGKGLEWVASISHDGIKKYHAEKV | |
| Heavy | WGRFTISRDNSKNTLYLQMNSLRPEDTALYYC | |
| | AKDLREDECEEWWSDYYDFGAQLPCAKSRGGL | |
| | VGIADNWGQGTMVTVSS | |
| CAP256V2LS: | QSVLTQPPSVSAAPGQKVTISCSGNTSNIGNN | 112 |
| Variable | FVSWYQQRPGRAPQLLIYETDKRPSGIPDRFS | |
| Light | ASKSGTSGTLAITGLQTGDEADYYCATWAASL | |
| | SSARVFGTGTQVIVLGQPKVNPTVTL | |

### III. PHARMACEUTICAL COMPOSITIONS AND FORMULATIONS

In some embodiments, the present disclosure provides pharmaceutical compositions. Such compositions comprise a prophylactically or therapeutically effective amount of an agent, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

In some embodiments, pharmaceutical compositions may comprise a peptide of interest. In preferred embodiments, the peptide of interest may comprise a sulfotyrosine residue at one or more positions. A therapeutic complex comprising the peptide of interest can be a combination of any therapeutic complexes with other chemical components, such as carriers, stabilizers, diluents, dispersing agents, suspending agents, thickening agents, and/or excipients. A pharmaceutical composition facilitates administration of the therapeutic complex to an organism.

Pharmaceutical formulations for administration can include aqueous solutions in water-soluble form. Suspensions of the active compound can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. The active ingredient can be in powder form for constitution with a suitable vehicle, for example, sterile pyrogen-free water, before use.

Pharmaceutical compositions comprising the peptide of interest can include at least one pharmaceutically acceptable carrier, diluent, or excipient and compounds as free-base or pharmaceutically acceptable salt form. Non-limiting examples of pharmaceutically-acceptable excipients suitable for use include binding agents, disintegrating agents, anti-adherents, anti-static agents, surfactants, antioxidants, coating agents, coloring agents, plasticizers, preservatives, suspending agents, emulsifying agents, anti-microbial agents, spheronization agents, and any combination thereof.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

A therapeutic complex comprising the peptide of interest can be conveniently formulated into pharmaceutical compositions composed of one or more pharmaceutically acceptable carriers. See e.g., Remington's Pharmaceutical Sciences, latest edition, by E. W. Martin Mack Pub. Co., Easton, PA, incorporated by reference in its entirety, which discloses typical carriers and conventional methods of preparing pharmaceutical compositions. Such carriers can be carriers for administration of compositions to humans and non-humans, including solutions such as sterile water, saline, and buffered solutions at physiological pH. Pharmaceutical compositions can also include one or more additional active ingredients such as antimicrobial agents, anti-inflammatory agents, and anesthetics.

Non-limiting examples of pharmaceutically acceptable carriers include saline, Ringer's solution, and dextrose solution. In some embodiments, the pH of the solution can be from about 5 to about 8 or can be from about 7 to about 7.5. Further carriers include sustained release preparations such as semipermeable matrices of solid hydrophobic polymers containing the therapeutic complex. The matrices can be in the form of shaped articles, for example, films, liposomes, microparticles, or microcapsules.

Non-limiting examples of pharmaceutically active agents suitable for combination with compositions include anti-infectives, i.e., aminoglycosides, antiviral agents, antimicrobials, anti-cholinergics/anti-spasmotics, antidiabetic agents, antihypertensive agents, anti-neoplastics, cardiovascular agents, central nervous system agents, coagulation modifiers, hormones, immunologic agents, immunosuppressive agents, and ophthalmic preparations.

In some embodiments, the pharmaceutical composition comprising a peptide containing sulfotyrosine comprises a therapeutically effective amount of a therapeutic complex herein in admixture with a pharmaceutically acceptable carrier and/or excipient, for example, saline, phosphate buffered saline, phosphate and amino acids, polymers, polyols, sugar, buffers, preservatives, and other proteins. Illustrative agents include octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringe″s and Han″s solutions, cysteine, arginine, carnitine, alanine, glycine, lysine, valine, leucine, polyvinylpyrrolidone, polyethylene, and glycol.

In some embodiments, a pharmaceutical formulation comprising the peptide of interest can comprise: (i) a therapeutic complex; (ii) a buffer; (iii) a non-ionic detergent; (iv) a tonicity agent; and (v) a stabilizer. In some embodiments, the pharmaceutical formulation is a stable liquid pharmaceutical formulation.

In some embodiments, a pharmaceutical formulation comprising the peptide of interest is a liquid formulation that can comprise about 5 mg/mL to about 150 mg/mL of the therapeutic complex, about 7.5 mg/mL to about 140 mg/mL of the therapeutic complex, about 10 mg/mL to about 130 mg/mL of the therapeutic complex, about 10 mg/mL to about 100 mg/mL of the therapeutic complex, about 20 mg/mL to about 80 mg/mL of the therapeutic complex, or about 30 mg/mL to about 70 mg/mL of the therapeutic complex. For example, a formulation of the present disclosure can comprise about 5 mg/mL, about 10 mg/mL, about 15 mg/mL, about 20 mg/mL, about 25 mg/mL, about 30 mg/mL, about 35 mg/mL, about 40 mg/mL, about 50 mg/mL, about 60 mg/mL, about 70 mg/mL, about 80 mg/mL, about 90 mg/mL, about 100 mg/mL, about 120 mg/mL, about 140 mg/mL, or about 150 mg/mL of a therapeutic complex.

In some embodiments, a pharmaceutical formulation comprising the peptide of interest can comprise a buffer. In some embodiments, the buffer serves to maintain a stable pH and to help stabilize a therapeutic complex disclosed herein. In some embodiments, the buffer or buffer system comprises at least one buffer that has a buffering range that overlaps fully or in part the range of pH 5.5-7.4. In some embodiments, the buffer has a pKa of about 6.2±0.5. In some embodiments, the buffer comprises a sodium phosphate buffer. In some embodiments, the sodium phosphate is present at a concentration of about 5 mM to about 15 mM, about 6 mM to about 14 mM, about 7 mM to about 13 mM, about 8 mM to about 12 mM, about 9 mM to about 11 mM, or about 10 mM.

In certain embodiments, the buffer system comprises sodium phosphate at 10 mM, at a pH of 6.2±0.3 or 6.1±0.3.

The pH can range from about 3 to about 12. The pH of the composition can be, for example, from about 3 to about 4, from about 4 to about 5, from about 5 to about 6, from about 6 to about 7, from about 7 to about 8, from about 8 to about 9, from about 9 to about 10, from about 10 to about 11, or from about 11 to about 12 pH units. The pH of the composition can be, for example, about 3, about 4, about 5, about 6, about 7, about 8, about 9, about 10, about 11, or about 12 pH units. The pH of the composition can be, for example, at least 3, at least 4, at least 5, at least 6, at least 7, at least 8, at least 9, at least 10, at least 11 or at least 12 pH units. The pH of the composition can be, for example, at most 3, at most 4, at most 5, at most 6, at most 7, at most 8, at most 9, at most 10, at most 11, or at most 12 pH units. A pharmaceutical formulation disclosed herein can have a pH of from about 5.5 to about 6.5. For example, a formulation of the present disclosure can have a pH of about 5.5, about 5.6, about 5.7, about 5.8, about 5.9, about 6.0, about 6.1, about 6.2, about 6.3, about 6.4, or about 6.5. In some embodiments, the pH is 6.2±0.3, 6.2+0.2, 6.2+0.1, about 6.2, or 6.2. If the pH is outside the range desired by the formulator, the pH can be adjusted by using sufficient pharmaceutically acceptable acids and bases.

In some embodiments, a pharmaceutical formulation comprising the peptide of interest can comprise a non-ionic detergent. In some embodiments, the non-ionic detergent is a nonionic polymer containing a polyoxyethylene moiety. In some embodiments, the non-ionic detergent is any one or more of polysorbate 20, poloxamer 188 or polyethylene glycol 3350. In some embodiments, the non-ionic detergent is polysorbate 20. In some embodiments, the non-ionic detergent is polysorbate 80. In some embodiments, a pharmaceutical formulation disclosed herein can contain about 0.01% to about 1% non-ionic detergent. For example, a formulation of the present disclosure can comprise about 0.0085%, about 0.01%, about 0.02%, about 0.03%, about 0.04%, about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.20%, about 0.21%, about 0.22%, about 0.23%, about 0.24%, about 0.25%, about 0.3%, about 0.4%, about 0.5%, about 0.6%, about 0.7%, about 0.8%, about 0.9%, about 1%, about 1.1%, about 1.15%, about 1.2%, about 1.25%, about 1.3%, about 1.35%, about 1.4%, about 1.45%, about 1.5%, about 1.55%, about 1.6%, about 1.65%, about 1.7%, about 1.75%, about 1.8%, about 1.85%, about 1.9%, about 1.95%, or about 2% polysorbate 20, polysorbate 80 or poloxamer 188.

In some embodiments, a pharmaceutical formulation comprising the peptide of interest can comprise a tonicity agent. In some embodiments, the tonicity agent is sodium chloride or potassium chloride. In some embodiments, the tonicity agent is sodium chloride. In some embodiments, sodium chloride is present at a concentration of about 5 mM to about 100 mM, about 10 mM to about 50 mM, or about 40 mM.

In some embodiments, a pharmaceutical formulation comprising the peptide of interest can comprise a stabilizer. In some embodiments, the stabilizer is a thermal stabilizer that can stabilize a therapeutic complex disclosed herein under conditions of thermal stress. In some embodiments, the stabilizer maintains greater than about 93% of the therapeutic complex in a native conformation when the solution containing the therapeutic complex and the thermal stabilizer is kept at about 45° C. for up to about 28 days. In some embodiments, the stabilizer prevents aggregation of the therapeutic complex and less than 4% of the therapeutic complex is aggregated when the solution containing the therapeutic complex and the thermal stabilizer is kept at about 45° C. for up to about 28 days. In some embodiments, the stabilizer maintains greater than about 96% of the therapeutic complex in a native conformation when the solution containing the therapeutic complex and the thermal stabilizer is kept at about 37° C. for up to about 28 days. In some embodiments, the stabilizer prevents aggregation of the therapeutic complex and less than about 2% of the therapeutic complex is aggregated when the solution containing the therapeutic complex and the thermal stabilizer is kept at about 37° C. for up to about 28 days.

In some embodiments, the thermal stabilizer is a sugar or sugar alcohol, for example, sucrose, sorbitol, glycerol, trehalose, or mannitol, or any combination thereof. In some embodiments, the stabilizer is a sugar. In some embodiments, the sugar is sucrose, mannitol or trehalose. In some embodiments, the stabilizer is sucrose. In some embodiments, a pharmaceutical formulation or ophthalmic formulation disclosed herein can comprise about 1% to about 20% sugar or sugar alcohol, about 2% to about 18% sugar or sugar alcohol, about 3% to about 15% sugar or sugar alcohol, about 4% to about 10% sugar or sugar alcohol, or about 5% sugar or sugar alcohol. For example, a pharmaceutical formulation or ophthalmic formulation of the present disclosure can comprise about 4%, about 5%, about 6%, about 7%, about 8%, about 9%, about 10%, about 11%, about 12%, about 13%, or about 14% sugar or sugar alcohol (e.g., sucrose, trehalose or mannitol). In some embodiments, the stabilizer is at a concentration of from about 1% w/v to about 20% w/v. In some embodiments, the stabilizer is sucrose at a concentration of from about 1% w/v to about 15% w/v, or from about 1% w/v to about 10% w/v. In some embodiments, the stabilizer is sucrose at a concentration of 5% w/v or about 5% w/v. In some embodiments, the stabilizer is sucrose at a concentration of 7.5% w/v or about 7.5% w/v. In some embodiments, the stabilizer is sucrose at a concentration of 10% w/v or about 10% w/v. In some embodiments, the stabilizer is sucrose at a concentration of 12.5% w/v or about 12.5% w/v. In some embodiments, the stabilizer is sucrose at a concentration of 15% w/v or about 15% w/v. In some embodiments, the stabilizer is sucrose at a concentration of 20% w/v or about 20% w/v.

A therapeutic complex comprising the peptide of interest can be, for example, an immediate release form or a controlled release formulation. An immediate release formulation can be formulated to allow the therapeutic complex to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some embodiments, a controlled release formulation is a delayed release form. A delayed release form can be formulated to delay a therapeutic complex's action for an extended period of time. A delayed release form can be formulated to delay the release of an effective dose of one or more therapeutic complexes, for example, for about 4, about 8, about 12, about 16, or about 24 hours.

A controlled release formulation can be a sustained release form. A sustained release form can be formulated to sustain, for example, the therapeutic complex's action over an extended period of time. A sustained release form can be formulated to provide an effective dose of any therapeutic complex described herein (e.g., provide a physiologically effective blood profile) over about 4, about 8, about 12, about 16, or about 24 hours.

A therapeutic complex comprising the peptide of interest can be produced by various methods in any quantity. For example, a therapeutic complex disclosed herein can be produced in an amount of about 1 microgram, about 1 milligram, about 1 gram, about 1 kilogram, or more.

Non-limiting examples of production methods include in vitro transcription methods, polymerase chain transcription (PCT), recombinant overexpression (e.g., in E. coli, R. sulfidophilum, or other in vitro systems), transfer RNA (tRNA) scaffold methods, enzymatic methods, chemical methods, solid-phase oligonucleotide synthesis, solid-phase chemical synthesis, ribozyme cleavage methods, T4 ligation methods, position-selective labeling of RNA (PLOR), T7 RNA polymerase in vitro methods, T3 RNA polymerase in vitro methods, SP6 RNA polymerase in vitro methods, phosphoramidite chemistry, cell-free nucleic acid expression methods, or a combination thereof.

Non-limiting examples of purification methods include precipitation and solvent extraction, ultracentrifugation, polyacrylamide gel electrophoresis (PAGE), liquid chromatography (e.g., reversed-phase ion-pairing HPLC (RP-IP-HPLC), ion-exchange HPLC (IE-HPLC), ion-exchange fast-performance liquid chromatography (IE-FPLC), affinity chromatography (e.g., systematic evolution of ligands by exponential enrichment (SELEX), and size-exclusion chromatography (SEC)), or a combination thereof. Purification methods can be used to achieve varying degrees of purity of a therapeutic complex disclosed herein, e.g., at least 80% purity, at least 85% purity, at least 90% purity, at least 91% purity, at least 92% purity, at least 93% purity, at least 94% purity, at least 95% purity, at least 96% purity, at least 97% purity, at least 98% purity, at least 99% purity, or at least 99.99%.

Activity of the therapeutic complex comprising the peptide of interest can be detected with various protein activity assays, such as western blot, flow cytometry, immunofluorescence, immunoprecipitation, ELISA, and the like. In some embodiments, an antibody such as an antibody-HRP conjugate or antibody fluorophore conjugate is used for the protein activity assays.

Methods for the preparation of compositions comprising the peptide of interest include formulating the therapeutic complex with one or more inert, pharmaceutically acceptable excipients or carriers to form a solid, semi-solid, or liquid composition. Solid compositions include, for example, powders, tablets, dispersible granules, capsules, cachets, and suppositories. Liquid compositions include, for example, solutions in which a therapeutic complex is dissolved, emulsions comprising a therapeutic complex, or a solution containing liposomes, micelles, nanoparticles, vesicles, microvesicles, or nanovesicles comprising the therapeutic complex as disclosed herein. Semi-solid compositions include, for example, gels, suspensions, and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically acceptable additives.

Compositions comprising the peptide of interest can be packaged as a kit. In some embodiments, the present disclosure provides a kit comprising a composition disclosed herein, and written instructions on use of the kit.

In some embodiments, properties and/or activities of agents, complexes, and compositions thereof can be characterized and/or assessed using various technologies available to those skilled in the art, e.g., biochemical assays, cell-based assays, animal models, clinical trials, etc. Those skilled in the art reading the present disclosure will readily appreciate that other technologies, e.g., in vitro models (e.g., cell lines) for various conditions, disorders, or diseases (e.g., viral infections), animal models for various conditions, disorders, or diseases (e.g., viral infections), clinical trials, etc. may be designed and/or utilized to assess provided technologies (e.g., agents, complexes, compounds, and compositions thereof, methods, etc.) in accordance with the present disclosure.

As appreciated by those skilled in the art, pharmaceutical compositions comprising the peptide of interest are useful for many purposes. In some embodiments, pharmaceutical compositions comprising the peptide of interest are useful for treating various conditions, disorders, or diseases, e.g., viral infections, e.g., HIV-1, in a subject. In some embodiments, technologies are useful for controlling and/or minimizing blood loss, e.g., in a system, e.g., in a subject, e.g., in a human.

In some embodiments, an effective amount of a pharmaceutical composition comprising the peptide of interest may be administered or delivered to a system. In some embodiments, a system comprises or is an in vitro system. In some embodiments, a system comprises or is an in vivo system. In some embodiments, a system comprises or is a cell. In some embodiments, a system comprises or is a population of cells. In some embodiments, a system comprises or is a tissue. In some embodiments, a system comprises or is an organ. In some embodiments, a system comprises or is an organism. In some embodiments, a system comprises or is a subject. In some embodiments, a system comprises or is a mammal, e.g., a mouse, rat, monkey, etc. In some embodiments, a system is a human.

Various conditions, disorders, or diseases may be treated with provided technologies (e.g., compositions, and methods). In some embodiments, a condition, disorder, or disease is a viral infection. In some embodiments, a condition, disorder, or disease is an HIV infection.

In some embodiments, the present disclosure provides methods for treating a condition, disorder, or disease in a subject comprising administering to the subject a therapeutically effective amount of a pharmaceutical composition comprising the peptide of interest. In some embodiments, the present disclosure provides methods for preventing a disease or disorder in a subject comprising administering to the subject a therapeutically effective amount of a pharmaceutical composition comprising the peptide of interest. In some embodiments, the present disclosure provides methods for treating an infection in a subject comprising administering to a subject a therapeutically effective amount of a pharmaceutical composition comprising the peptide of interest.

In some embodiments, a pharmaceutical composition comprising the peptide of interest may be utilized in combination with another therapy, e.g., an additional therapeutic agent. In some embodiments, a method for treating provided herein further comprises administering to a subject a therapeutically effective amount of one or more additional therapeutic agents. In some embodiments, one or more additional therapeutic agents comprise one or more therapeutic agents described herein. In some embodiments, one or more additional therapeutic agents comprise one or more agents comprising a pharmaceutical composition comprising the peptide of interest. Effective combination therapy may be achieved with a single composition or pharmacological formulation that includes both agents, or with two distinct compositions or formulations, administered at the same time, wherein one composition includes a composition of this invention, and the other includes the second agent(s). Alternatively, the therapy may precede or follow the other agent treatment by intervals ranging from minutes to months.

Non-limiting examples of such combination therapy include combination of one or more compositions comprising the peptide of interest with another anti-inflammatory agent, a chemotherapeutic agent, radiation therapy, an antidepressant, an antipsychotic agent, an anticonvulsant, a mood stabilizer, an anti-infective agent, an antihypertensive agent, a cholesterol-lowering agent or other modulator of blood lipids, an agent for promoting weight loss, an antithrombotic agent, an agent for treating or preventing cardiovascular events such as myocardial infarction or stroke, an antidiabetic agent, an agent for reducing transplant rejection or graft-versus-host disease, an anti-arthritic agent, an analgesic agent, an anti-asthmatic agent or other treatment for respiratory diseases, or an agent for treatment or prevention of skin disorders.

In practicing the methods of treatment or use provided herein, therapeutically effective amounts of the therapeutic complex comprising the peptide of interest are administered in pharmaceutical compositions to a subject having a disease or condition to be treated. In some embodiments, the subject is a mammal such as a human. Non-limiting examples of possible subjects for administration include the following. Subjects can be humans, non-human primates such as chimpanzees, and other apes and monkey species; farm animals such as cattle, horses, sheep, goats, and swine; domestic animals such as rabbits, dogs, and cats; and laboratory animals including rats, mice, and guinea pigs. A subject can be of any age. Subjects can be, for example, elderly adults, adults, adolescents, pre-adolescents, children, toddlers, infants, and neonates. A therapeutically effective amount can vary widely depending on the severity of the disease, the age and relative health of the subject, the potency of the therapeutic complex used, and other factors.

A pharmaceutical composition comprising the peptide of interest can be administered in a therapeutically-effective amount by various forms and routes including, for example, parenteral, intravenous injection, intravenous infusion, subcutaneous injection, subcutaneous infusion, intramuscular injection, intramuscular infusion, intradermal injection, intradermal infusion, intraperitoneal injection, intraperitoneal infusion, intracerebral injection, intracerebral infusion, subarachnoid injection, subarachnoid infusion, intraocular injection, intraspinal injection, intrasternal injection, endothelial administration, local administration, intranasal administration, intrapulmonary administration, rectal administration, intraarterial administration, intrathecal administration, inhalation, intralesional administration, intradermal administration, epidural administration, absorption through epithelial or mucocutaneous linings (e.g., oral mucosa, rectal and intestinal mucosa), intracapsular administration, subcapsular administration, intracardiac administration, transtracheal administration, subcuticular administration, subarachnoid administration, subcapsular administration, intraspinal administration, or intrasternal administration.

A pharmaceutical composition comprising the peptide of interest can be administered in a local manner, for example, via injection of the therapeutic complex directly into an organ, optionally in a depot or sustained release formulation or implant. A pharmaceutical composition can be provided in the form of a rapid release formulation, in the form of an extended-release formulation, or in the form of an intermediate release formulation. A rapid release form can provide an immediate release. An extended-release formulation can provide a controlled release or a sustained delayed release.

A therapeutic complex comprising the peptide of interest can be administered before, during, or after the occurrence of a disease or condition, and the timing of administering the composition containing a therapeutic complex can vary. For example, a therapeutic complex can be used as a prophylactic and can be administered continuously to subjects with a propensity to conditions or diseases in order to lessen or reduce a likelihood of the occurrence of the disease or condition. A therapeutic complex/composition comprising the peptide of interest can be administered to a subject during or as soon as possible after the onset of the symptoms. The administration of a therapeutic complex comprising the peptide of interest can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A therapeutic complex comprising the peptide of interest can be administered as soon as is practical after the onset of a disease or condition is detected or suspected, and for a length of time necessary for the treatment of the disease, such as, for example, from about 1 month to about 3 months. In some embodiments, the length of time a therapeutic complex can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, or about 25 years.

A therapeutic complex comprising the peptide of interest can be administered at any interval desired. The administration of the therapeutic complex can have regular or irregular dosing schedules to accommodate either the person administering the therapeutic complex or the subject receiving the therapeutic complex. For example, the therapeutic complex can be administered twice a day, once a day, five times a week, four times a week, three times a week, two times a week, once a week, once every two weeks, once every three weeks, once every four weeks, once a month, once every five weeks, once every six weeks, once every eight weeks, once every two months, once every twelve weeks, once every three months, once every four months, once every six months, once a year, or less frequently. In some embodiments, administration is every other week.

The amount administered can be of the same amount in each dose or the dosage can vary between doses. For example, a first amount can be administered in the morning and a second amount can be administered in the evening.

Multiple therapeutic complexes comprising the peptide of interest can be administered in any order or simultaneously. If simultaneously, the therapeutic complexes can be provided in a single, unified form, or in multiple forms, for example, as multiple separate injections or infusions. The therapeutic complexes can be packed together or separately, in a single package or in a plurality of packages. One or all of the therapeutic complexes can be given in multiple doses. If not simultaneous, the timing between the multiple doses can vary to as much as about a month. The length of treatment can vary for each subject. Amounts effective for this use can vary based on the severity and course of the disease or condition, previous therapy, the subject's health status, weight, and response to the drugs, and the judgment of the treating physician.

A therapeutic complex comprising the peptide of interest can be administered via subcutaneous or intravenous injection. The volume of an injection can be about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, about 2 mL, about 2.1 mL, about 2.2 mL, about 2.3 mL, about 2.4 mL, about 2.5 mL, about 2.6 mL, about 2.7 mL, about 2.8 mL, about 2.9 mL, or about 3 mL.

A therapeutic complex comprising the peptide of interest can be administered at a dosage of about 0.0001 mg/kg to about 1000 mg/kg, about 0.001 mg/kg to about 100 mg/kg, about 0.01 mg/kg to about 100 mg/kg, about 0.01 mg/kg to about 20 mg/kg, about 0.02 mg/kg to about 7 mg/kg, about 0.03 mg/kg to about 5 mg/kg, about 0.05 mg/kg to about 3 mg/kg, about 0.1 mg/kg to about 50 mg/kg, about 0.1 mg/kg to about 0.5 mg/kg, about 0.2 mg/kg to about 0.6 mg/kg, about 0.3 mg/kg to about 0.7 mg/kg, about 0.4 mg/kg to about 0.8 mg/kg, about 0.1 mg/kg to about 0.9 mg/kg, about 0.01 mg/kg to about 50 mg/kg, about 0.1 mg/kg to about 10 mg/kg, about 1 mg/kg to about 10 mg/kg, about 5 mg/kg to about 10 mg/kg, about 1 mg/kg to about 5 mg/kg, or about 3 mg/kg to about 7 mg/kg by mass of the subject.

A therapeutic complex comprising the peptide of interest can be administered in any amount necessary or convenient. For example, a therapeutic complex described herein can be administered in an amount from about 0.05 mg to about 300 mg, about 0.1 mg to about 300 mg, about 0.1 mg to about 200 mg, about 0.1 mg to about 100 mg, about 0.05 mg to about 1.5 mg, about 0.1 mg to about 1.5 mg, about 0.05 mg to about 1 mg, about 1 mg to about 1.5 mg, about 0.5 mg to about 6 mg, about 1 mg to about 4 mg, about 2 mg to about 10 mg, about 10 mg to about 30 mg, about 30 mg to about 50 mg, about 50 mg to about 70 mg, about 70 mg to about 100 mg, or about 0.1 mg to about 1 mg, about 0.05 mg, about 0.06 mg, about 0.07 mg, about 0.08 mg, about 0.09 mg, about 0.1 mg, about 0.11 mg, about 0.12 mg, about 0.13 mg, about 0.14 mg, about 0.15 mg, about 0.16 mg, about 0.17, mg, about 0.18 mg, about 0.19 mg, about 0.2 mg, about 0.21 mg, about 0.22 mg, about 0.23 mg, about 0.24 mg, about 0.25 mg, about 0.26 mg, about 0.27, mg, about 0.28 mg, about 0.29 mg, about 0.3 mg, about 0.31 mg, about 0.32 mg, about 0.33 mg, about 0.34 mg, about 0.35 mg, about 0.36 mg, about 0.37, mg, about 0.38 mg, about 0.39 mg, about 0.4 mg, about 0.41 mg, about 0.42 mg, about 0.43 mg, about 0.44 mg, about 0.45 mg, about 0.46 mg, about 0.47, mg, about 0.48 mg, about 0.49 mg, about 0.5 mg, about 0.51 mg, about 0.52 mg, about 0.53 mg, about 0.54 mg, about 0.55 mg, about 0.56 mg, about 0.57, mg, about 0.58 mg, about 0.59 mg, about 0.6 mg, about 0.61 mg, about 0.62 mg, about 0.63 mg, about 0.64 mg, about 0.65 mg, about 0.66 mg, about 0.67, mg, about 0.68 mg, about 0.69 mg, about 0.7 mg, about 0.71 mg, about 0.72 mg, about 0.73 mg, about 0.74 mg, about 0.75 mg, about 0.76 mg, about 0.77, mg, about 0.78 mg, about 0.79 mg, about 0.8 mg, about 0.81 mg, about 0.82 mg, about 0.83 mg, about 0.84 mg, about 0.85 mg, about 0.86 mg, about 0.87, mg, about 0.88 mg, about 0.89 mg, about 0.9 mg, about 0.91 mg, about 0.92 mg, about 0.93 mg, about 0.94 mg, about 0.95 mg, about 0.96 mg, about 0.97, mg, about 0.98 mg, about 0.99 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg about 200 mg, about 210 mg, about 220 mg, about 230 mg, about 240 mg, about 250 mg, about 260 mg, about 270 mg, about 280 mg, about 290 mg, or about 300 mg per dose for a subject by any route of administration.

Pharmaceutical compositions comprising the peptide of interest can be in unit dosage forms suitable for single administration of precise dosages. In unit dosage form, the formulation is divided into unit doses containing appropriate quantities of one or more therapeutic complexes. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for parenteral injection can be presented in unit dosage form, for example, in ampoules, or in multi-dose containers with a preservative.

### IV. DEFINITIONS

The use of the word “a” or “an,” when used in conjunction with the term “comprising” in the claims and/or the specification may mean “one,” but it is also consistent with the meaning of “one or more,” “at least one,” and “one or more than one.”

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10% unless the context indicates otherwise. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or amino acid residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has, for example, at least 85 percent sequence identity, preferably at least 90 to 95 percent sequence identity, more preferably at least 99 percent sequence identity, as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

“Variant” with respect to a peptide or peptide, may indicate that the peptide or peptide differs in amino acid sequence by the insertion, deletion, or conservative substitution of amino acids, but retains at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art (Kyte et al., J Mol. Biol. 157:105-132, 1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of +2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within 12 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a pharmaceutical substance means that amount of the pharmaceutical substance which, when administered to a subject or patient for treating or preventing a disease, is an amount sufficient to effect such treatment or prevention of the disease.

The terms “peptide” and “polypeptide” shall be taken to include a single chain, i.e., a series of contiguous amino acids linked by peptide bonds or a series of chains covalently or non-covalently linked to one another (i.e., a peptide complex). For example, the series of peptide chains can be covalently linked using a suitable chemical linker or a disulfide bond, for example. Examples of non-covalent bonds include hydrogen bonds, ionic bonds, Van der Waals forces, and hydrophobic interactions.

The terms “peptide”, “polypeptide” or “peptide chain” will be understood from the foregoing paragraph to mean a series of contiguous amino acids linked by peptide bonds.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio. One example of compositions which are pharmaceutically acceptable include those compositions, materials, and/or dosage forms that have been designated by the United States Food and Drug Administration (US FDA) as having a status of generally regarded as safe (GRAS).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### V. EXAMPLES

### Example 1—Discovery of Tyrosine Sulfotransferase using a Sequence Similarity Network

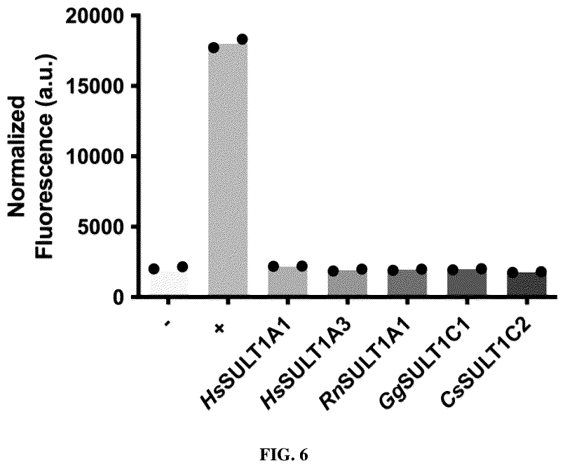

To identify the enzyme responsible for sulfation of cytoplasmic tyrosine, the inventors focused on cytosolic sulfotransferases. Based on their reported substrate specificities, SULT1A1 and SULT1A3 from Homo sapiens, SULT1A1 from Rattus norvegicus, and SULT1C1 from Gallus gallus were first examined (Allali-Hassani et al., 2007; Jendresen & Nielsen, 2019), all of which are known to recognize multiple phenolic substrates. To explore the activity of these sulfotransferases towards tyrosine, a green fluorescent protein assay was used (Chen et al., 2018; Chen et al., 2019). These four sulfotransferase genes were codon-optimized for Escherichia coli and cloned into the pBad vector. To generate a suppression plasmid for sulfotyrosine incorporation, a pUltra-sulfotyrosine plasmid encoding the engineered Methanococcus jannaschii tyrosyl-tRNA synthetase (sulfotyrosineRS) and its corresponding MjtRNATyrCUA was used (Liu & Schultz, 2006; Schwessinger et al., 2016). The suppressor plasmid (pUltra-sulfotyrosine) was used to suppress the amber codon (Asp134TAG) within a sfGFP variant encoded by the pLei-sfGFP134TAG plasmid in the presence of sulfotyrosine. Expression of full-length sfGFP was carried out in LB medium for 16 hours in parallel with controls BL21(DE3) harboring pUltra-sulfotyrosine, pLei-sfGFP134TAG and pBad-Empty in the presence and absence of exogenously fed 1 mM sulfotyrosine. As expected, sfGFP was expressed in the presence of 1 mM sulfotyrosine fed in controls cells (FIG. 6). Unfortunately, none of these four sulfotransferases led to sfGFP expression, indicating the failure of the biosynthesis of sulfotyrosine. To circumvent the limited substrate range of the reported sulfotransferases, the full repertoire of protein sequence diversity in nature was accessed by using a sequence similarity network (SSN, FIG. 1B) (Copp et al., 2018). SSNs provide an effective way to visualize and analyze the relatedness of massive protein sequences on the basis of similarity thresholds of amino acid sequence (Gerlt et al., 2015). The inventors initially created an SSN with EFI-ESI based on rat SULT1A1 as an input sequence, since its cognate substrate p-coumaric acid is similar to tyrosine (FIGS. 1B and 1C) (Jendresen & Nielsen, 2019). An alignment score of 110 was set to limit the edges and a sequence identity of 80% was used to generate representative nodes, which resulted in a final SSN of 391 representative enzyme sequences. Interestingly, it was found that human SULT1C2, whose substrate is tyramine, was in a different cluster of the SSN (FIGS. 1B and 1C) (Allali-Hassani et al., 2007). It was hypothesized that enzymes with high sequence similarity to rat SULT1A1 and human SULT1C2 would possess the ability to carry out the sulfation of tyrosine. To test this hypothesis, 27 sequences were selected from the SSN based on their proximity to both RnSULT1A1 and HsSULT1C2. These selected genes were cloned into the pBad vector and tested with the green fluorescent protein assay. A 2.5-fold increase in fluorescence was observed for cells expressing A0A091VQH7 compared to cells not given exogenous sulfotyrosine, suggesting that sulfotyrosine was biosynthesized intracellularly and incorporated into sfGFP proteins (FIG. 1D). A0A091VQH7 is a putative sulfotransferase from Nipponia nippon, with over 90% sequence identity with SULT1C1 reported in other species. Thus, A0A091VQH7 was named as NnSULT1C1 hereafter (Blanchard et al., 2004).

### Example 2—Molecular Basis of NnSULT1C1 Action in the Sulfation of Tyrosine

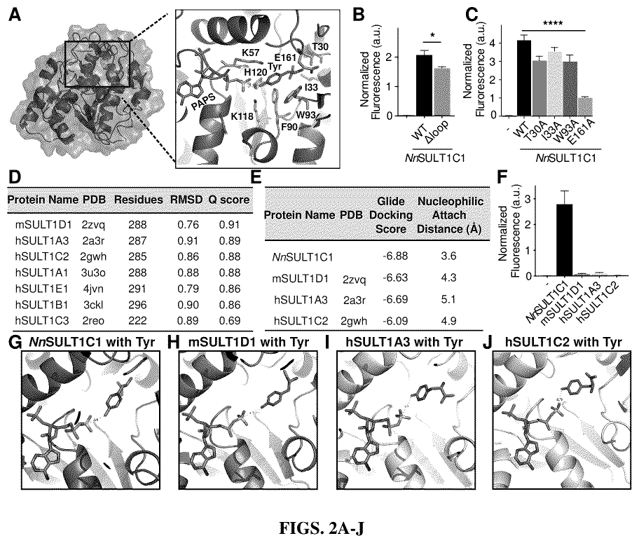

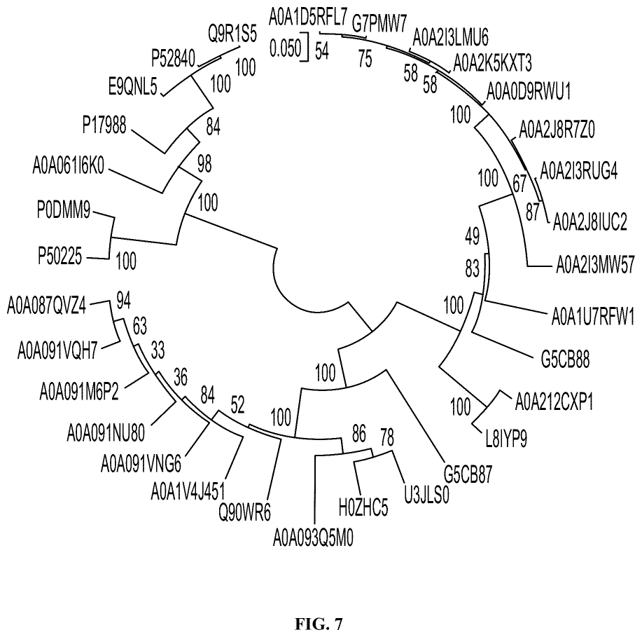

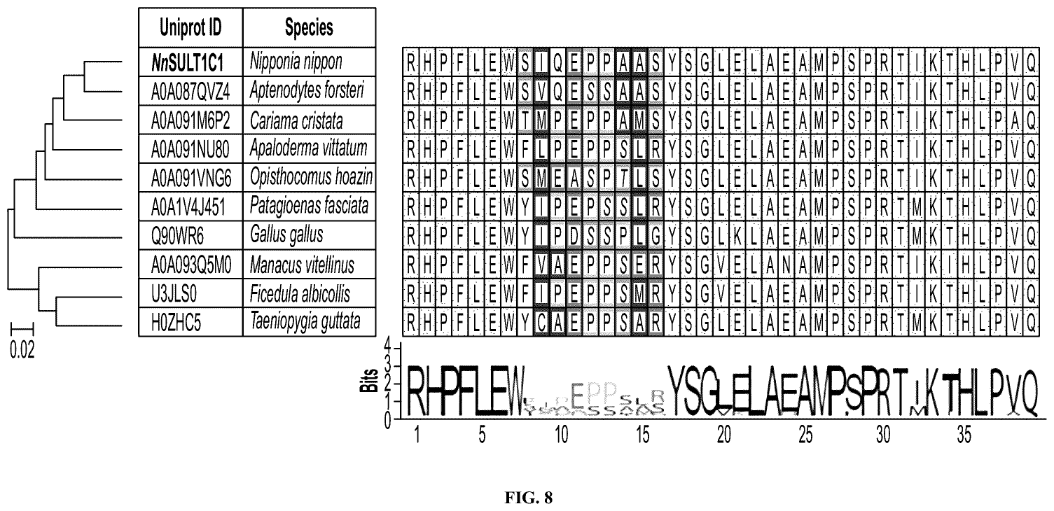

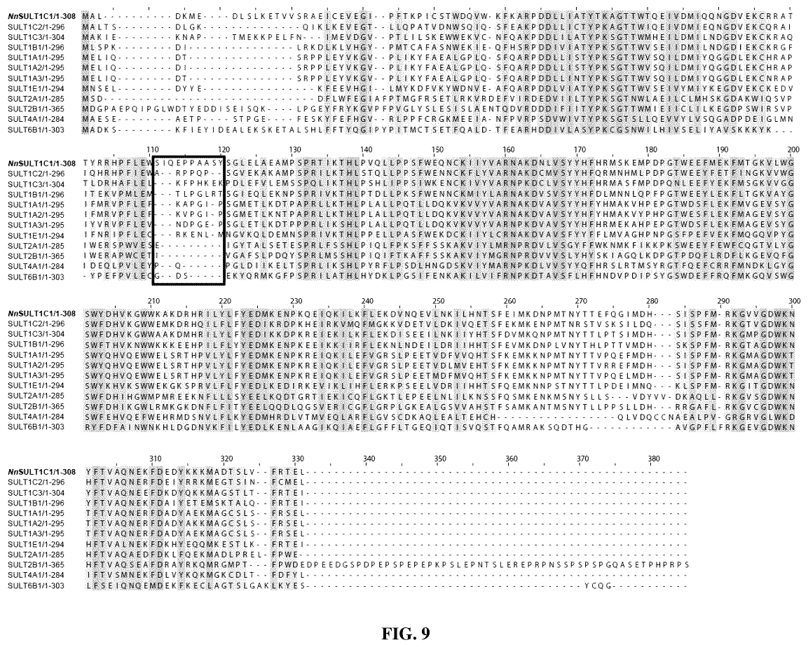

To explore the origin of the unique tyrosine specificity of NnSULT1C1 among all the sulfotransferases tested, the phylogenetic relationships of the enzymes were first analyzed. Sulfotransferase amino acid sequences were used to generate a phylogenetic tree using the unweighted pair group method with arithmetic mean (UPGMA) by MEGA X software package (FIG. 7) (Schlee, 1975). The tree was subdivided into three major subfamilies, among which NnSULT1C1 falls into subfamily I containing bird sulfotransferases. Most sequences from subfamilies II and III are derived from rodent and primate groups, respectively. To further analyze the molecular basis of the unique tyrosine specificity of NnSULT1C1, a multiple sequence alignment (MSA) of all sequences within subfamily I of the phylogenetic tree was performed (FIG. 8). This sequence alignment revealed that most regions of NnSULT1C1, including the PAPS-binding site, are highly conserved throughout the tree except for a highly variable region corresponding to NnSULT1C1 residues 94-103 (SIQEPPAASY) and residues likely involved in substrate binding pocket (Varin et al., 1997; HirschmaNn et al., 2014). To explore the contribution of this highly variable region and these residues to substrate binding, the structure of NnSULT1C1 was predicted via Alphafold 2. Alphafold 2 is a machine learning approach that has been shown to predict protein structure with a high degree of accuracy (Jumper et al., 2021; Tunyasuvunakool et al., 2021; Jin et al., 2020a; Jin et al., 2020b). More than 90% of the residues in the predicted NnSULT1C1 structure show Local Distance Difference Test (IDDT) values over 90, indicating they have a significant likelihood of very high accuracy in the predicted structure. Similar to the structures of other cytosolic sulfotransferases, the overall predicted structure of NnSULT1C1 is composed of classical α/β motifs (FIG. 2A) (Berger et al., 2011; Lu et al., 2005 This structure includes a β sheet surrounded by α-helices, giving rise to a narrow substrate-binding site (FIG. 2A) (Bidwell et al., 1999). It was found that the highly variable region (94-102 residues) of NnSULT1C1 constitutes a loop for the substrate entry, which also aligns with the substrate entry loop of human cytosolic sulfotransferase (FIG. 9) (Allali-Hassani et al., 2007). The deletion of this loop on NnSULT1C1, however, only results in 22% decrease of its activity to produce fluorescent protein with sulfotyrosine. (FIG. 2B)

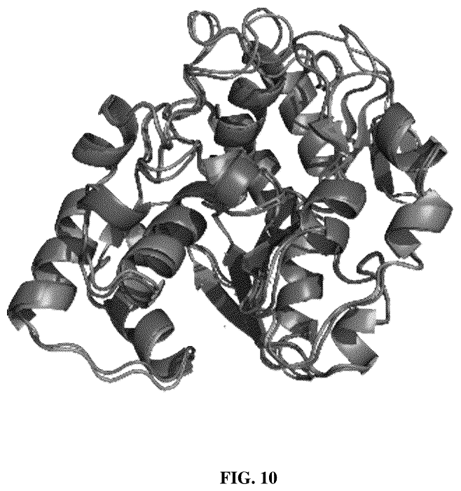

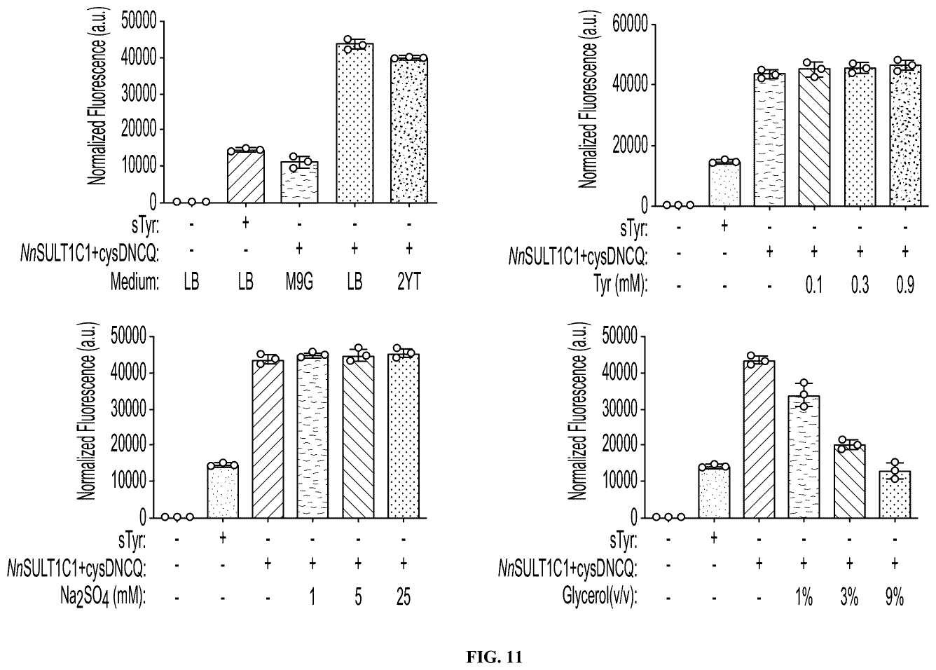

To further explore the other residues involved in substrate binding of NnSULT1C1, protein-ligand docking was performed using Glide v8.1 in Schrödinger software package v2018.4 (Fiesner et al., 2004). The Tyr was docked to the NnSULT1C1 using OPLS_3 force field and the lowest energy pose was monitored (Banks et al., 2005). For each docking experiment, 200 maximum output poses for each protein were set and Emodel energy was used for ranking the top 50 poses. The docking structure suggests that the a-amino group of Tyr is stabilized by NnSULT1C1 residues Glu161, Thr30, Ile33, and Trp93. The π-π stacking interactions between Tyr and Phe90 are likely to improve the packing interaction (FIG. 2A). The phenolic hydroxy group of Tyr is in the proper Lys-Lys-His catalytic site to engage in sulfuryl transfer. The His120 residue serves as a catalytic base that can remove the proton from Tyr and stabilize the transition state. The Lys57 and Lys118 residues interact with and stabilize the sulfuryl group of PAPS and the phenolic hydroxy group of Tyr, respectively. To validate the contribution of these residues interacting with Tyr on NnSULT1C1 activity, Thr30, Ile33, Trp93, and Glu161 were mutated to alanine separately. Alanine mutation at Thr30, Trp93, or Glu161 significantly decreased the activity of NnSULT1C1 (FIG. 2C). Among these residues, the E161A mutation exhibits the largest decrease in activity, confirming its important interaction with Tyr. To further explore whether other sulfotransferases may also carry out the tyrosine sulfation, a structure similarity search was performed using PDBeFold. Based on the Q score, the three proteins with structures most similar to NnSULT1C1 are mouse SULT1D1 (pdb: 2zvq), human SULT1A3 (pdb: 2a3r) and human SULT1C2 (pdb: 2gwh, FIG. 2D). The overall secondary structure of NnSULT1C1 aligned well with 2zvq, which indicates its structural consistency with the other cytosolic sulfotransferases. (FIG. 10) To further illustrate the unique specificity of NnSULT1C1 for Tyr, dockings of Tyr to the most similar sulfotransferases, including mSULT1D1, hSULT1A3, and hSULT1C2, were carried out using Glide v8.1 in the Schrödinger software package v2018.4 following the same method of Tyr docking used for NnSULT1C1 (Friesner et al., 2004). Docking of Tyr to NnSULT1C1 exhibits the lowest Glide Docking score of −6.88 and the closest distance between the phenolic hydroxyl group and PAPS sulfonate. (FIG. 2E) This result is consistent with the optimal ability of NnSULT1C1, to generate sulfotyrosine-containing sfGFP in the green fluorescent protein assay among all tested sulfotransferases (FIG. 2F). The key step of the sulfotransfer reaction involves an S_{N}2-type nucleophilic attack on the PAPS sulfonate by the phenoxide of Tyr. Compared with mSULT1D1, hSULT1A3, and hSULT1C1, the docking of Tyr in NnSULT1C1 results in the closest distance (3.6 Å) between the sulfur atom of PAPS and the phenolic hydroxyl group (FIG. 2G-J). Furthermore, the acceptor phenolic hydroxyl group of Tyr lies on the backside of the S—O bond of PAPS in the Tyr docking structure with NnSULT1C1, indicating a more proper orientation for the nucleophilic attack (FIG. 2G).

### Example 3—Biosynthesis and Genetic Encoding of Sulfotyrosine in Escherichia coli

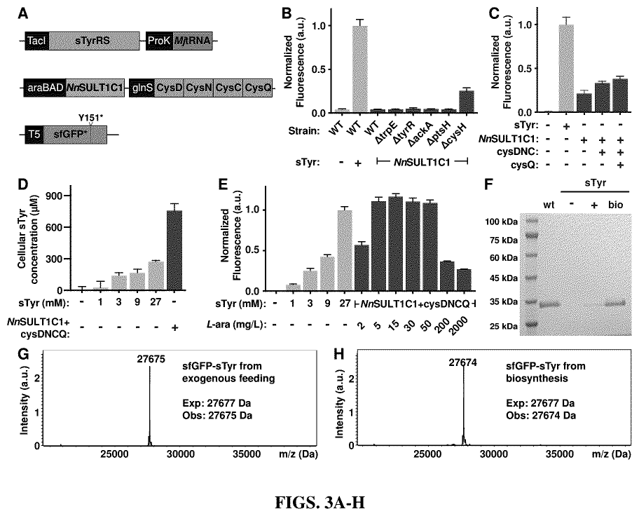

Having identified NnSULT1C1 as a functional tyrosine sulfotransferase, the inventors explored whether the biosynthesized sulfotyrosine can be genetically incorporated into proteins in E. coli in response to the amber codon. An initial goal was to increase sulfotyrosine production in these cells in order to optimize its availability for incorporation into proteins. Since NnSULT1C1 utilizes tyrosine and PAPS for producing sulfotyrosine, sulfotyrosine production was quantified in five knockout E. coli cell lines in which the gene knockout has been shown to improve the yield of either tyrosine or PAPS in E. coli (Zhu et al., 2014; Wei et al., 2016; Bang et al., 2016; Chu et al., 2018). To evaluate the effect of knocking out these genes on the biosynthesis of sulfotyrosine, the suppression plasmid pUltra-sulfotyrosine, reporter plasmid pET22b-T5-sfGFP151TAG, and the biosynthesis plasmid pEvol-NnSULT1C1 was transformed into wild type E. coli BW25113 or knockout strains (FIG. 3A). The expression of sfGFP with sulfotyrosine at position 151 (sfGFP-sulfotyrosine) was carried out in LB medium for 18 h. It was found that knockout of the cysH gene significantly improved the production of sulfotyrosine-containing sfGFP, compared to that seen in the wildtype BW25113 strain (FIG. 3B). CysH encodes the PAPS sulfotransferase responsible for degradation of PAPS to 3′-phosphoadenosine-5′-phosphate (PAP). This observation of enhanced sfGFP-sulfotyrosine production in BW25113ΔcysH is consistent with the previous report that knockout of the cysH gene can increase cellular PAPS concentration and the production of sulfated products in E. coli (Chu et al., 2018; Badri et al., 2019). Next, the inventors examined whether manipulation of PAPS synthetic and recycling pathways in E. coli could enhance intracellular PAPS levels. The gene cysDNC encoding adenosine-5′-triphosphate (ATP) sulfurylase and adenosine 5′-phosphosulfate (APS) kinase was first amplified to increase the intracellular level of PAPS, followed by the introduction of the gene cycQ encoding adenosine-3′,5′-diphosphate (PAP) nucleotidase for PAP recycling (Jendresen & Nielsen, 2019; Badri et al., 2019; Neuwald et al., 1992; Spiegelberg et al., 1999). It was found that cells with both PAPS synthesis and recycling pathways exhibited the largest increase in fluorescence, suggesting a higher expression level of sfGFP containing sulfotyrosine (FIG. 3C). To examine the contribution of the biosynthetic pathway to intracellular sulfotyrosine concentration, the intracellular sulfotyrosine concentrations in cells was measured when sulfotyrosine was either biosynthesized or delivered via exogenous feeding. Since a high concentration of exogenous ncAA has been shown to be associated with higher protein production using the Genetic Code Expansion technology, the effect of exogenous sulfotyrosine concentrations was screened to up to 27 mM, higher than all reported concentration added externally (Li et al., 2018; Liu & Schultz, 2006; Liu et al., 2009; Liu et al., 2008). The cellular concentration of sulfotyrosine in cells endowed with the sulfotyrosine biosynthetic pathway is 756.3 μM, which is 28-fold higher than that from cells exogenously fed with 1 mM sulfotyrosine and higher even than in cells fed with 27 mM (FIG. 3D). Consistent with these intracellular levels of sulfotyrosine, in the context of Genetic Code Expansion technology, endogenous biosynthesis of sulfotyrosine results in much higher sfGFP-sulfotyrosine expression than that produced via exogenous feeding (FIG. 3E). Predictably, the NnSULT1C1 expression level also has a significant influence on the production of sfGFP-sulfotyrosine. Since it was found that NnSULT1C1 expression induced by 15 mg/L L-arabinose yielded the highest production of sfGFP-sulfotyrosine, 15 mg/L L-ara was consistently used for induction of the sulfotyrosine biosynthetic pathway (FIG. 3E). Other variables were also screened for sfGFP-sulfotyrosine expression, including expression medium, Tyr addition, SO42− addition, glycerol addition, which did not optimize the expression of sfGFP-sulfotyrosine. (FIG. 11) To further investigate the efficiency and specificity of incorporation of biosynthesized sulfotyrosine in these autonomous E. coli cells, sfGFP-sulfotyrosine proteins derived from exogenously fed sY and from biosynthesized sY were purified by Ni2+-NTA affinity chromatography and characterized by SDS-PAGE and ESI-MS. Intact sfGFP was only expressed after exogenous sulfotyrosine feeding or after induction of sulfotyrosine biosynthesis. The yield of sfGFP-sulfotyrosine derived from biosynthetic sulfotyrosine is 5.67 mg/L sfGFP-sulfotyrosine under the optimal condition, compared with 1.5 mg/L sfGFP-sulfotyrosine produced by feeding with 1 mM exogenous sulfotyrosine (FIG. 3F). The mass of sfGFP-sulfotyrosine produced from biosynthetic sulfotyrosine was 27,674 Da, which is in good agreement with the calculated mass. (FIGS. 3G and 3H).

### Example 4—Biosynthesis and Genetic Encoding of Sulfotyrosine in Mammalian Cells

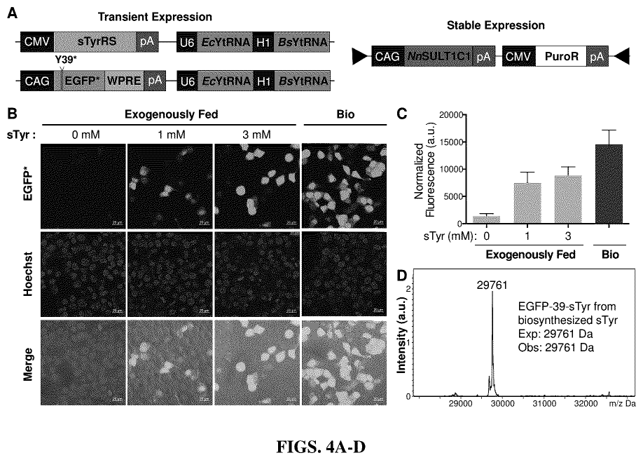

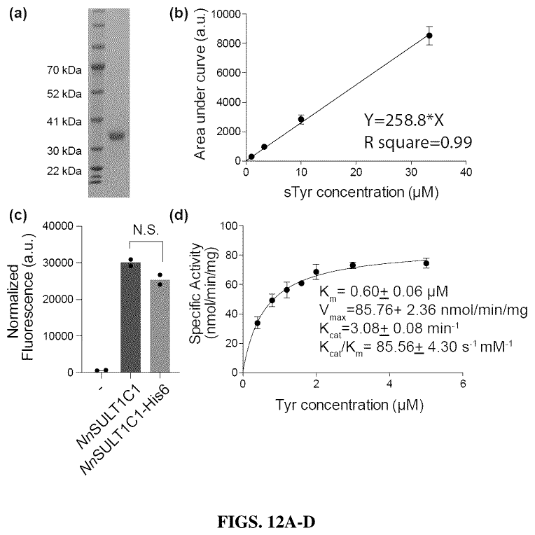

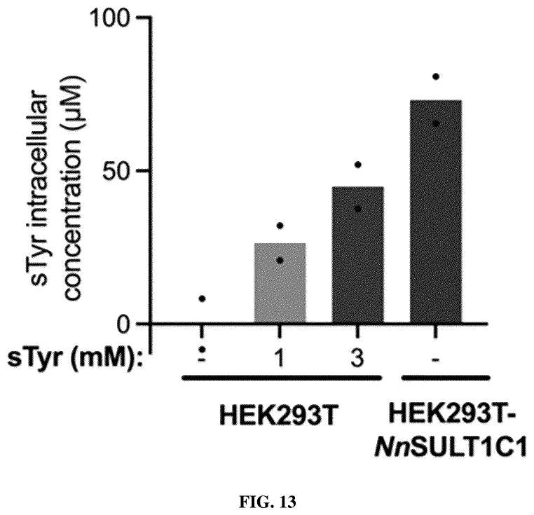

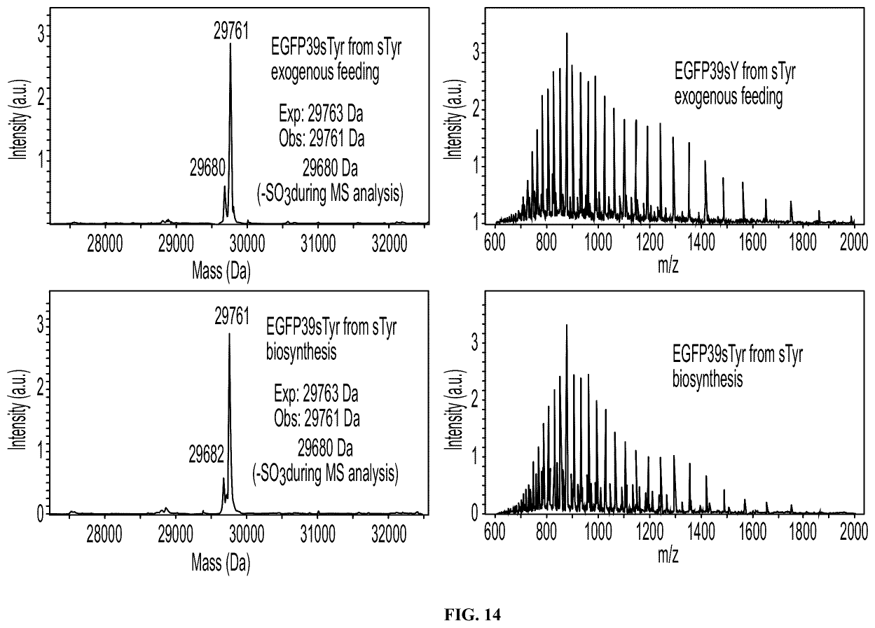

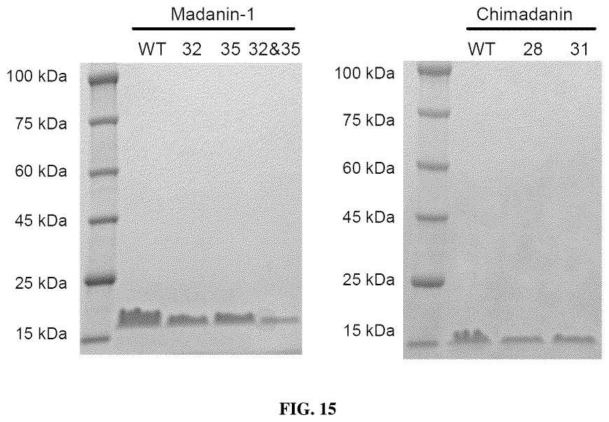

To promote the efficient expression of mammalian proteins sulfated on specific tyrosines, mammalian cells were created and were equipped with both sulfotyrosine biosynthetic and translational machinery. To generate mammalian cells capable of biosynthesizing sulfotyrosine, a piggybac system was used to stably integrate NnSULT1C1 into the genome of HEK293T cells, yielding the HEK293T-NnSULT1C1 cell line (FIG. 4A) (Yusa et al., 2011). The EcTyrRS/tRNA pair was used to construct pAcBac2.tR4-sulfotyrosineRS/EGFP*, containing EGFP with a stop codon at position 39 as well as two copies of E. coli and Bacillus stearothermophilus tRNACUATyr (FIG. 4A) (Chatterjee et al., 2013). To evaluate the function of NnSULT1C1 in mammalian cells, pAcBac2.tR4-sulfotyrosineRS/EGFP* was transfected into HEK293T and HEK293T-NnSULT1C1 cells, which were then incubated in the presence or absence of exogenous sulfotyrosine. The expression of EGFP was monitored by confocal microscopy at 2 days after transfection. As expected, the addition of 1 mM sulfotyrosine to HEK293T cells resulted in moderate expression of full-length EGFP, while minimal EGFP fluorescence was observed in the absence of sulfotyrosine addition (FIG. 4B). Gratifyingly, higher expression of EGFP was observed in HEK293T-NnSULT1C1 cells without exogenous sulfotyrosine addition than that seen in HEK293T cells fed with 3 mM sulfotyrosine. In addition to confocal imaging, flow cytometry was used to quantify expression levels of EGFP in cells fed with exogenous sulfotyrosine and in cells biosynthesizing sulfotyrosine. As shown in FIG. 4C, significantly higher EGFP fluorescence was observed in HEK293T-NnSULT1C1 cells endowed with sulfotyrosine biosynthetic capability than in HEK293T cells fed with 3 mM sulfotyrosine. As a direct evidence of sulfotyrosine biosynthesis in mammalian cells, cellular sulfotyrosine concentration in HEK293T-NnSULT1C1 is more than that in HEK293T cells fed with 3 mM sulfotyrosine. (FIG. 12) The fidelity of site-specific incorporation of sulfotyrosine was evaluated by mass spectral analysis of purified sulfotyrosine-containing EGFP proteins. The observed mass was 29,761 Da, consistent with the calculated mass of EGFP with sulfotyrosine at position 39 and observed mass of EGFP39sulfotyrosine purified from HEK293T with external sulfotyrosine addition (FIG. 4D). These results demonstrate that the creation of mammalian cells autonomously able to biosynthesize sulfotyrosine and incorporate it into proteins significantly enhances the expression level of sulfotyrosine-containing protein in mammalian cells.

### Example 5—Using Completely Autonomous Sulfotyrosine Biosynthetic Cells to Synthesize Potent Thrombin Inhibitors with Site-Specific Sulfation

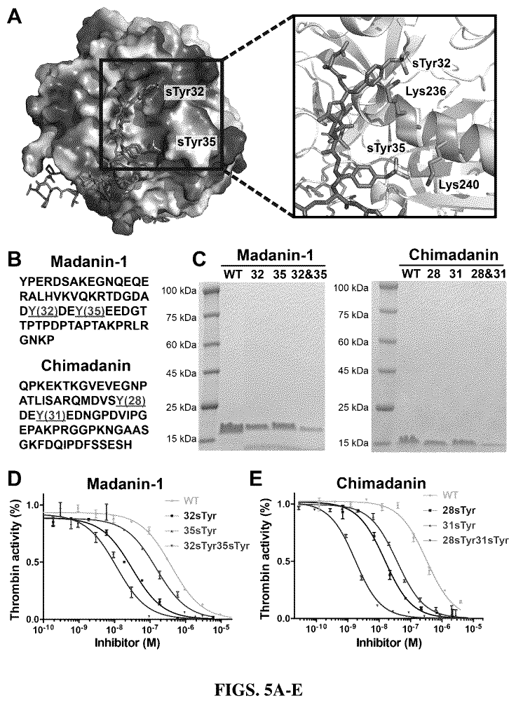

Tyrosine sulfation of madanin-1 and chimadanin significantly increases their affinities for thrombin by promoting strong electrostatic interactions with positively-charged residues (FIG. 5A). To explore the generation sulfotyrosine-containing thrombin inhibitors using cells endowed with autonomous sulfotyrosine biosynthetic machinery, both madanin-1 and chimadanin identified in the salivary gland of Haemaphysalis longicornis were chosen (FIG. 5B) (Thompson et al., 2017; Nakajima et al., 2006). As shown in FIG. 5A, sulfation of madanin-1 converts Tyr32 and Tyr35 to negative residues, thus enhancing madanin-1's direct electrostatic interaction with the F-amino groups of K236 and K240 located within the exosite II site of thrombin. To express the site-specifically sulfated thrombin inhibitors, plasmids encoding the thrombin inhibitor were constructed and substituted with amber codons at either or both of the indicated Tyr sites. sulfotyrosine-containing inhibitors were expressed by transforming ΔcysH BW25113 cells with pEvol-NnSULT1C1-cysDNCQ, pUltra-sulfotyrosine, and a plasmid encoding the thrombin inhibitor. In parallel, the ΔcysH BW25113 cells lacking the sulfotyrosine biosynthetic systems but exogenously fed with 3 mM sulfotyrosine were utilized. The site-specific sulfation of madanin-1 and chimadanin was further validated using ESI-MS analysis (FIG. 5C).

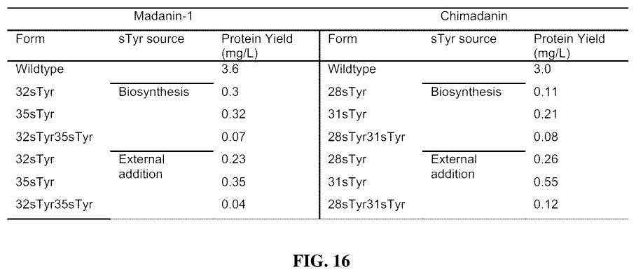

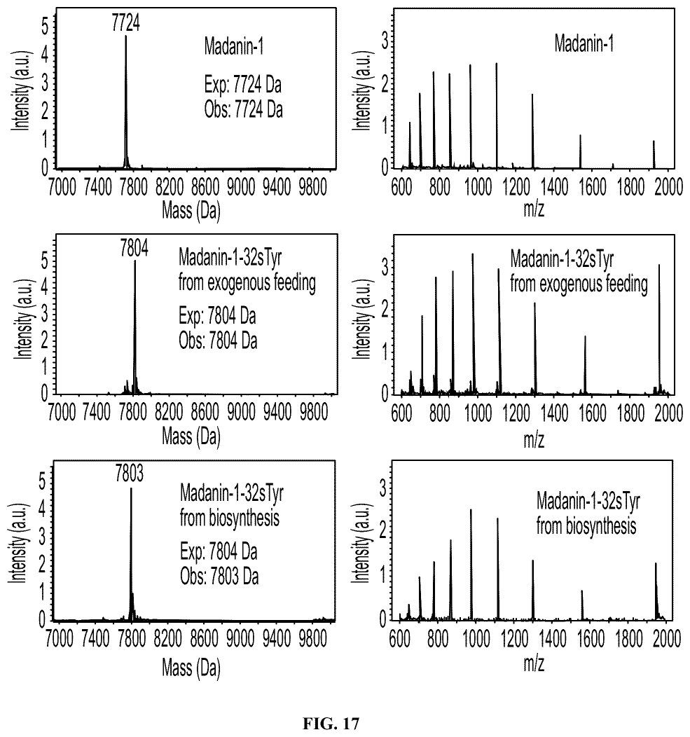

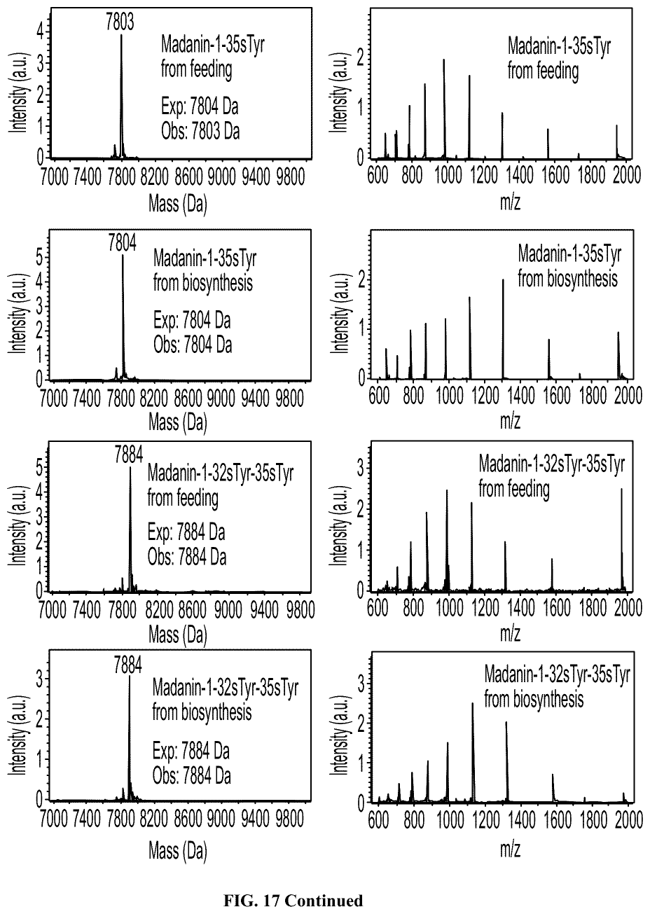

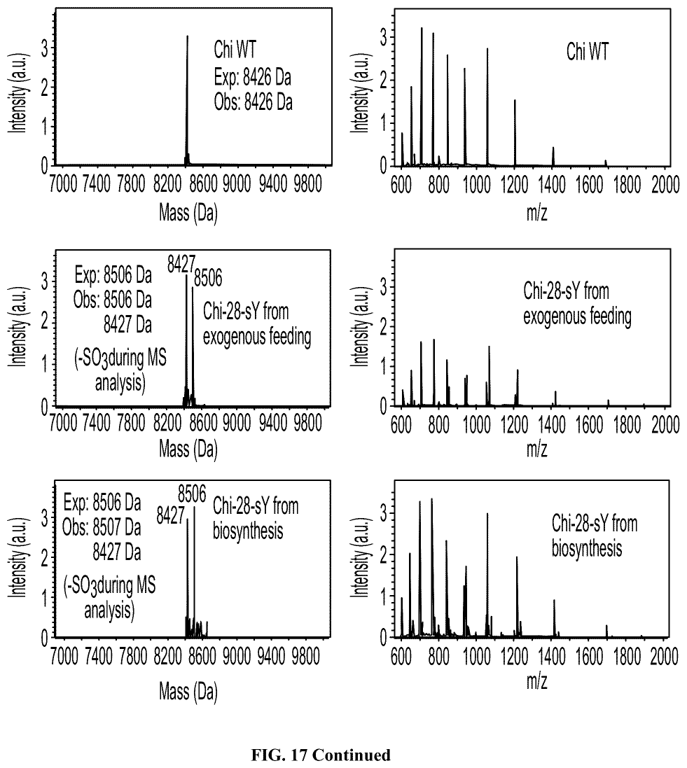

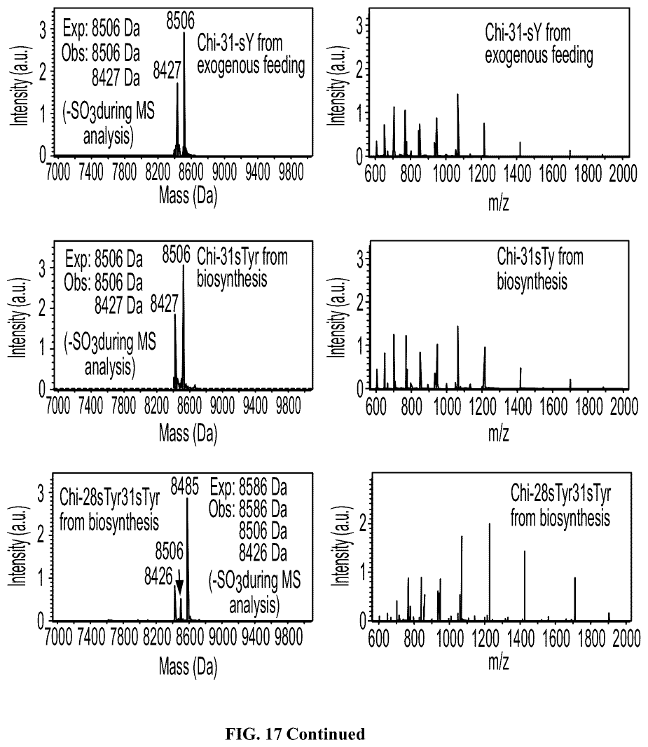

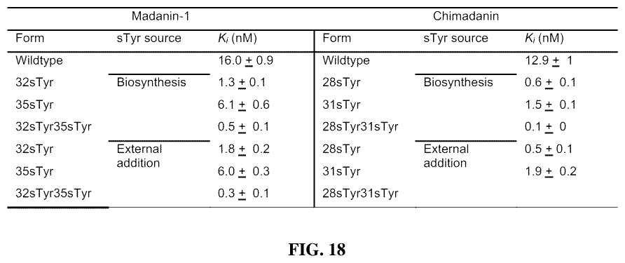

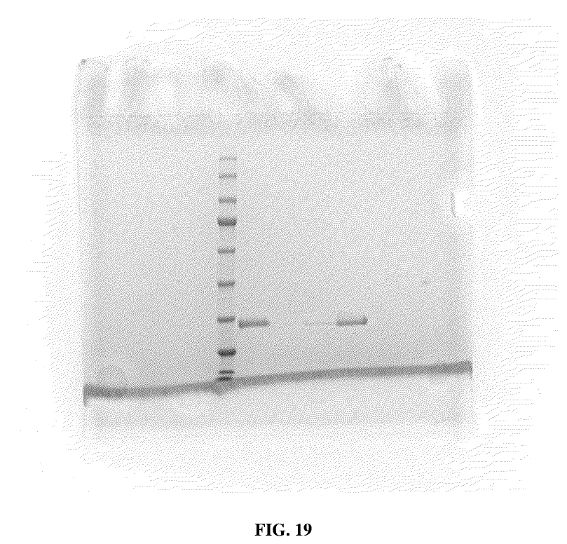

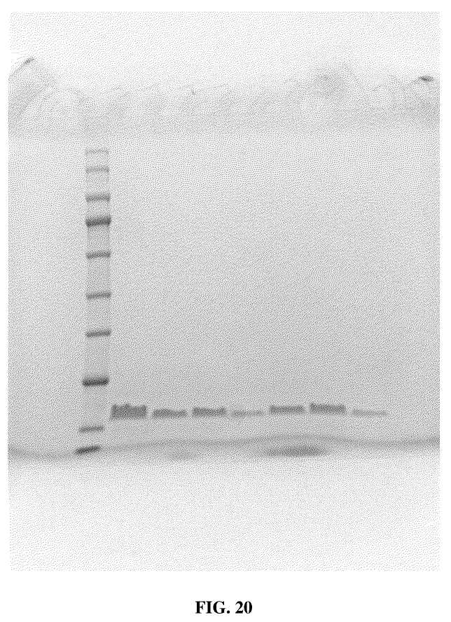

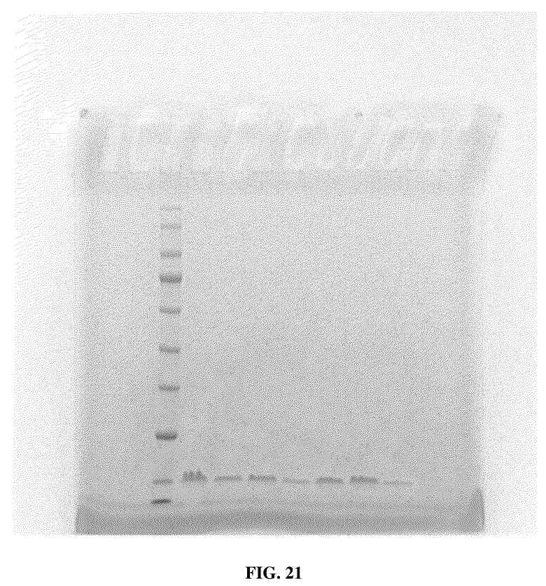

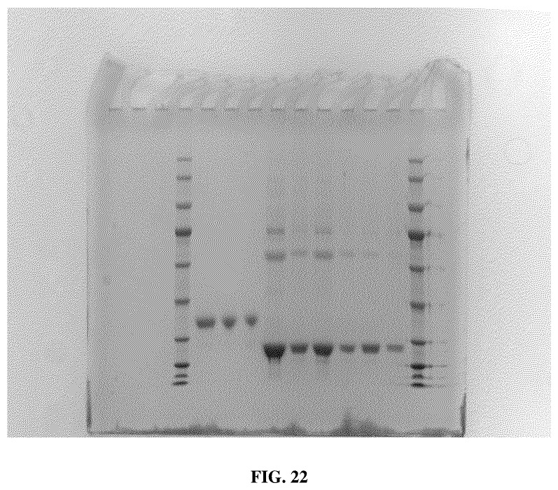

To test the thrombin inhibiting activity of the wild type inhibitors and their sulfotyrosine-containing mutants, chromogenic thrombin amidolytic activity assays were performed in the presence of a range of concentrations of each inhibitor. Compared with wildtype madanin-1 (Kᵢ=16.0+0.9 nM), incorporation of a single sulfotyrosine at either Tyr32 (Kᵢ=1.3+0.1 nM) or Tyr35 (Kᵢ=6.1+0.6 nM) position significantly enhanced its inhibition of thrombin (FIG. 5D). Madanin-1 mutants sulfated at both Tyr32 and Tyr35 exhibited the highest potency (Kᵢ=0.5+0.1 nM) against thrombin activity (FIG. 5D and FIG. 16). Following a similar trend, incorporating a single biosynthesized sulfotyrosine at either Tyr28 or Tyr31 of chimadanin yields more potent inhibition of thrombin activity (Kᵢ=0.6+0.1 nM and 1.5+0.1 nM, respectively) than achieved with wildtype chimadanin (Kᵢ=12.9+0.1 nM, FIG. 5E and FIG. 16). Double sulfation of chimadanin at both Tyr28 and Tyr31 further improved its Kᵢ to 0.1 nM, consistent with the madanin-1 study. Furthermore, sulfotyrosine-containing thrombin inhibitors prepared using cells with completely autonomous sulfotyrosine biosynthetic machinery are more potent than chemically synthesized ones (Thompson et al., 2017). This may be due to the fact that co-translational folding is more efficient than that achieved via chemical synthesis. These data demonstrate the advantages of producing therapeutic proteins with site-specific sulfotyrosine modifications using completely autonomous cells with the ability to biosynthesize and genetically encode sulfotyrosine.

### Example 6—Materials and Methods

Plasmid DNA preparation was carried out with the GenCatch™ Plasmid DNA Miniprep Kit and GenCatch™ Advanced Gel Extraction Kit. M9-glucose minimal medium contain M9 salt (6.78 g/L Na₂HPO₄, 3 g/L KH₂PO₄, 1 g/L NH₄Cl, 0.5 μg/L NaCl), heavy metal solution (1 μg/L CuSO·5H₂O, 4 μg/L MnCl·4H₂O, 4 μg/L ZnCl₂, 1.2 μg/L FeSO·5H2O), 1 mM MgSO₄, 0.1 mM CaCl₂, 5 μg/mL Thiamine, 300 μM Leucine 4 μM D-Biotin, Glucose (4 g/L). Table 2 lists the oligonucleotides used herein.

TABLE 2

| | | | SEQ |
|---|---|---|---|
| Oligonu- | | | ID |
| cleotide | Sequence (5′-3′) | Note | NO |
| Da343 | cataaaatcacctcaacctctagatacc | | 2 |
| Da344 | taagtcgaccgatgcccttgag | | 3 |
| Da326 | gaattcattaaagaggagaaattacatATGGAATTGATTCAAG | HsSU | 4 |
| | ATACGAGCCGCC | LT1A1 | |
| Da327 | gctctcaagggcatcggtcgacttaTCACAGCTCACTACGAA | HsSU | 5 |
| | AGCTAAGCGAG | LT1A1 | |
| Da328 | gaattcattaaagaggagaaattacatATGGAGTTCTCTCGC | HsSU | 6 |
| | CCTCCACTTGTGC | LT1A3 | |
| Da329 | gctctcaagggcatcggtcgacttaTCACAATTCGCAACGAA | HsSU | 7 |
| | ACTTGAAATCG | LT1A3 | |
| Da330 | gaattcattaaagaggagaaattacatATGGAACTTATCCAG | RnSU | 8 |
| | GATACCTCCCGTC | LT1A1 | |
| Da331 | gctctcaagggcatcggtcgacttaTCAAAGCTCCGAGCGAA | RnSU | 9 |
| | ACGACAGTGAAC | LT1A1 | |
| Da332 | gaattcattaaagaggagaaattacatATGGCTCTGGACAAG | GgSU | 10 |
| | ATGGAAAACTTG | LT1C1 | |
| Da333 | gctctcaagggcatcggtcgacttaTCAAAGTTCCATACGGA | GgSU | 11 |
| | AAACCAAGCTAG | LT1C1 | |
| Da345 | gtatctagaggttgaggtgattttATGGCCTTAACATCTGACC | O0033 | 12 |
| | TTGGTAAG | 8 | |
| Da346 | gctctcaagggcatcggtcgacttaTCAGAGTTCCATGCAGA | O0033 | 13 |
| | AGTTGATGC | 8 | |
| Da347 | gtatctagaggttgaggtgattttATGGCCTTAACTTCAGAGT | A0A1 | 14 |
| | TAGGGAAAC | D5RF | |
| | | L7 | |
| Da348 | gctctcaagggcatcggtcgacttaTCACAGCTCCATACAGA | A0A1 | 15 |
| | AGTTAATACTTGTG | D5RF | |
| | | L7 | |
| Da349 | gtatctagaggttgaggtgattttATGGCTCAGGTTCCTGAAT | A0A1 | 16 |
| | TATCGAAACCG | U7RF | |
| | | W1 | |
| Da350 | gctctcaagggcatcggtcgacttaTCACAGTTTCATACAAAA | A0A1 | 17 |
| | GTTAATCGAAGTGC | U7RF | |
| | | W1 | |
| Da351 | gtatctagaggttgaggtgattttATGCTTTTAATCAGCACATA | A0A1V | 18 |
| | TGCAAAAGCG | 4J451 | |
| Da352 | gctctcaagggcatcggtcgacttaTCATTACAGTTCTGTAC | A0A1V | 19 |
| | GGAAAACAAGTGAAG | 4J451 | |
| Da353 | gtatctagaggttgaggtgattttATGATTGAGCAAAACGGGG | A0A2I | 20 |
| | ACGTG | 3LMU | |
| | | 6 | |
| Da354 | gctctcaagggcatcggtcgacttaTCACAGTTCCATACAAA | A0A2I | 21 |
| | AATTGATCGAGGTC | 3LMU | |
| | | 6 | |
| Da355 | gtatctagaggttgaggtgattttATGGCATTAACATCTGAGC | A0A2I | 22 |
| | TTGGTAAGC | 3MW5 | |
| | | 7 | |
| Da356 | gctctcaagggcatcggtcgacttaTCACAGTTCCATGCAGA | A0A2I | 23 |
| | AGTTAATACTTGTCC | 3MW5 | |
| | | 7 | |
| Da357 | gtatctagaggttgaggtgattttATGGATATGATTGAGCAGA | A0A2I | 24 |
| | ACGGCG | 3RUG | |
| | | 4 | |
| Da358 | gctctcaagggcatcggtcgacttaTCACAGCTCCATGCAGA | A0A2I | 25 |
| | AATTAATTGAAGTACC | 3RUG | |
| | | 4 | |
| Da359 | gtatctagaggttgaggtgattttATGGCGTTAACCTCGGACT | A0A2J | 26 |
| | TGG | 8IUC2 | |
| Da360 | gctctcaagggcatcggtcgacttaTCACAGTTCCATACAAA | A0A2J | 27 |
| | AGTTAATAGCCGTCC | 8IUC2 | |
| Da361 | gtatctagaggttgaggtgattttATGGCTCTGACTTCTGAAC | A0A2J | 28 |
| | TGGGGAAAC | 8R7Z0 | |
| Da362 | gctctcaagggcatcggtcgacttaTCACAGCTCCATACAAA | A0A2J | 29 |
| | AATTAATTGCAGTGC | 8R7Z0 | |
| Da363 | gtatctagaggttgaggtgattttATGGCATTAACAAGCGAGT | A0A2K | 30 |
| | TGGGGAAG | 5KXT3 | |
| Da364 | gctctcaagggcatcggtcgacttaTCACAGCTCCATGCAAA | A0A2K | 31 |
| | AGTTAATGCTTGTG | 5KXT3 | |
| Da365 | gtatctagaggttgaggtgattttATGGCTCTCGACAAAATGG | A0A09 | 32 |
| | AGGAC | 1VQH | |
| | | 7 | |
| Da366 | gctctcaagggcatcggtcgacttaTCACAGCTCAGTGCGAA | A0A09 | 33 |
| | AGACCAAC | 1VQH | |
| | | 7 | |
| Da367 | gtatctagaggttgaggtgattttATGGCCCTTATCACTGCAG | A0A21 | 34 |
| | GTACTC | 2CXP | |
| | | 1 | |
| Da368 | gctctcaagggcatcggtcgacttaTCACAGTTCTGTGCAAA | A0A21 | 35 |
| | AATTGATGCTGGTAC | 2CXP | |
| | | 1 | |
| Da369 | gtatctagaggttgaggtgattttAACATGGAGCTTATCAAAG | A0A06 | 36 |
| | ACATTTCGCG | 1I6K0 | |
| Da370 | gctctcaagggcatcggtcgacttaTCACAGGTTGCACCGAA | A0A06 | 37 |
| | ATTTGAGG | 1I6K0 | |
| Da371 | gtatctagaggttgaggtgattttATGGCGCAAAATCCAAGCA | E9QN | 38 |
| | ACATG | L5 | |
| Da372 | gctctcaagggcatcggtcgacttaTCAAATTTGACAACGAA | E9QN | 39 |
| | ATGTGAAATCGCAACCG | L5 | |
| Da373 | gtatctagaggttgaggtgattttATGGAACCACTGCGGAAG | P5284 | 40 |
| | CC | 0 | |
| Da374 | gctctcaagggcatcggtcgacttaTCAAATTTGGCACCGAA | P5284 | 41 |
| | AAGTGAAGTCG | 0 | |
| Da375 | gtatctagaggttgaggtgattttATGGCCCAGAACCCATCTA | Q9R1 | 42 |
| | ACATG | S5 | |
| Da376 | gctctcaagggcatcggtcgacttaTCAAATTTGACACCGGA | Q9R1 | 43 |
| | ATGTGAAGTCAC | S5 | |
| Da377 | gtatctagaggttgaggtgattttATGGCGGGCGAAGATCAC | G5CB | 44 |
| | AC | 87 | |
| Da378 | gctctcaagggcatcggtcgacttaTCAAATTTCAGTGCGGA | G5CB | 45 |
| | ATGTAAGGGTAC | 87 | |
| Da379 | gtatctagaggttgaggtgattttATGCGGAAACCTGAGCTG | G5CB | 46 |
| | GAG | 88 | |
| Da380 | gctctcaagggcatcggtcgacttaTCACAGTTCGAGGCAAA | G5CB | 47 |
| | ACCGG | 88 | |
| Da381 | gtatctagaggttgaggtgattttATGGCCCTCACTAGCGAAC | G7PM | 48 |
| | TTG | W7 | |
| Da382 | gctctcaagggcatcggtcgacttaTCACAGTTCCATACAGA | G7PM | 49 |
| | AGTTAATGGACGTG | W7 | |
| Da383 | gtatctagaggttgaggtgattttATGGCTTTAACTACCGCGG | L8IYP | 50 |
| | GTAC | 9 | |
| Da384 | gctctcaagggcatcggtcgacttaTCACAGTTCGGTGCAGA | L8IYP | 51 |
| | AGTTAATACTTGTC | 9 | |
| Da425 | gtatctagaggttgaggtgattttATGGCTTTGGATAAGATGG | A0A08 | 52 |
| | AAGACCTCTC | 7QVZ | |
| | | 4 | |
| Da426 | gctctcaagggcatcggtcgacttaTCAGAGTTCTGTGCGGA | A0A08 | 53 |
| | AGACTACG | 7QVZ | |
| | | 4 | |
| Da427 | gtatctagaggttgaggtgattttATGGCGCTGGACAAAATGA | A0A09 | 54 |
| | AGGAC | 1M6P | |
| | | 2 | |
| Da428 | gctctcaagggcatcggtcgacttaTCACTCCATACGAAAGA | A0A09 | 55 |
| | CCAGAGATGTG | 1M6P | |
| | | 2 | |
| Da429 | gtatctagaggttgaggtgattttATGCGTATGGAAGATCTTT | A0A09 | 56 |
| | CGCTGAAATAC | 1VNG | |
| | | 6 | |
| Da430 | gctctcaagggcatcggtcgacttaTCACAATTCCATGCGGA | A0A09 | 57 |
| | AGACTAACG | 1VNG | |
| | | 6 | |
| Da433 | gtatctagaggttgaggtgattttATGTGCAATGTGTTCCAGA | U3JLS | 58 |
| | TCACTACCG | 0 | |
| Da434 | gctctcaagggcatcggtcgacttaTCACAACTCTGCCCGAA | U3JLS | 59 |
| | ACACC | 0 | |
| Da435 | gtatctagaggttgaggtgattttATGGTAGACAAAATGAAAG | A0A09 | 60 |
| | ACCTCTCACTC | 3Q5M | |
| | | 0 | |
| Da436 | gctctcaagggcatcggtcgacttaTCATAATTCCATGCGGA | A0A09 | 61 |
| | AAACCAACGAG | 3Q5M | |
| | | 0 | |
| Da437 | gtatctagaggttgaggtgattttATGCTGGCCATGGACAAGA | H0ZH | 62 |
| | TGAAAG | C5 | |
| Da438 | gctctcaagggcatcggtcgacttaTCACAATTCCATACGGA | H0ZH | 63 |
| | AGACGAGGC | C5 | |
| Da439 | gtatctagaggttgaggtgattttATGTCTGGCACTACATGGA | A0A09 | 64 |
| | CTCAGG | 1NU80 | |
| Da440 | gctctcaagggcatcggtcgacttaTCAAAGTTCCGTCCGGA | A0A09 | 65 |
| | AAACAAGTG | 1NU80 | |
| Da443 | gcatgctcgagcagctcag | | 66 |
| Da444 | agatctaattcctcctgttagcccaaaaaaacg | | 67 |
| Da441 | tgggctaacaggaggaattagatctATGGCTCTCGACAAAAT | | 68 |
| | GGAGGAC | | |
| Da442 | cgaccctgagctgctcgagcatgcaGAAGACAGTCATAAGT | | 69 |
| | GCGGCGA | | |
| Da445 | atgggattcctcaaagcgtaaacaacgtataac | | 70 |
| Da446 | gtttacgctttgaggaatcccatATGGATCAAATACGACTTAC | | 71 |
| | TCACCTGCG | | |
| Da447 | cgaccctgagctgctcgagcatgcTCAGGATCTGATAATATC | | 72 |
| | GTTCTGTCTCAACAG | | |
| Da448 | TCAGGATCTGATAATATCGTTCTGTCTCAACAG | | 73 |
| Da449 | gacagaacgatattatcagatcctgaTAAGTTAACACCGCTC | | 74 |
| | ACAGAGACGAG | | |
| Da450 | cgaccctgagctgctcgagcatgcTTAGTAAATAGACACTCT | | 75 |
| | GAACCCCGGATTC | | |
| Da454 | gtcgaccatcatcatcatcatcattgagtttaaac | | 76 |
| Da462 | TCACTATAGGGAGACCCAAGCTGGCTAGCGCCA | | 77 |
| | CCATGGCGAGTTCCAATCTGATTAAGC | | |
| Da463 | GAGTTAAAGTCGACTTAACGCGTTGAATTCTTATA | | 78 |
| | CAGGTCCTTTCCAGCAAATGAGAC | | |
| Da556 | attcattaaagaggagaaattacatATGCAGCCTAAGGAAAA | | 79 |
| | AACAAAAGGTGTAG | | |
| Da557 | gagtccaagctcagctaattaagcttATGAGATTCAGAACTGA | | 80 |
| | AGTCAGGAATCTGATC | | |
| Da558 | attcattaaagaggagaaattacatATGTATCCAGAACGGGA | | 81 |
| | CTCCG | | |
| Da559 | gagtccaagctcagctaattaagcttCGGTTTGTTGCCCCGA | | 82 |
| | AGG | | |
| Da584 | TTACGACGAGtagGAGGACAACGC | | 83 |
| Da585 | CTAACGTCCATCTGACGGGCG | | 84 |
| Da586 | GGACGCGGATtagGATGAATATGAGGAAG | | 85 |
| Da587 | CCGTCGGTACGTTTCTGCACC | | 86 |
| Da655 | GGCAAAGAATTGCAAGTTTGTACAAAAAAGCAGG | | 87 |
| | CTGCCACCATGGCGCTTGACAAAATGGAAGAC | | |
| Da656 | GCCTGCACCTGAGGATCACCACTTTGTACAAGAA | | 88 |
| | AGCTGGGTTTAaagctctgtccgaaagacgagagaag | | |
| Da661 | TCCGCGTCCCCGTCGGTA | | 89 |
| Da662 | TTACGATGAAtagGAGGAAGACGGGACGAC | | 90 |
| Da663 | TTAGGATGAAtagGAGGAAGACGGGACGAC | | 91 |
| Da664 | ATCTGACGGGCGGAAATGAGCG | | 92 |
| Da665 | GGACGTTAGTtagGACGAGTACGAGGACAACG | | 93 |
| Da666 | GGACGTTAGTtagGACGAGTAGGAGGACAACG | | 94 |
| Da687 | attcattaaagaggagaaattacatATGGCTCTCGACAAAAT | | 95 |
| | GGAGGAC | | |
| Da688 | gagtccaagctcagctaattaagctTTAGTGGTGGTGGTGGT | | 96 |
| | GGTGCAGCTCAGTGCGAAAGACCAAC | | |
| Da858 | CGGCACCCGTTCCTCGAATGGTCTGGGCTTGAAT | | 97 |
| | TAGCGGAGGC | | |
| Da859 | CCATTCGAGGAACGGGTGCC | | 98 |
| Da871 | GAAGTCGAAGGTATCCCGTTCGCGAAGCCTATTT | | 99 |
| | GTAGTACGTGGGATCAAGTG | | |
| Da872 | GAACGGGATACCTTCGACTTCGCAG | | 100 |
| Da873 | GAAGGTATCCCGTTCACTAAGCCTGCGTGTAGTA | | 101 |
| | CGTGGGATCAAGTGTGGAAATTC | | |
| Da874 | AGGCTTAGTGAACGGGATACCTTCG | | 102 |
| Da875 | GCCGGCACCCGTTCCTCGAAGCGTCAATCCAGG | | 103 |
| | AGCCACCGGCT | | |
| Da876 | TTCGAGGAACGGGTGCCGGC | | 104 |
| Da877 | CTATCACTTTCACCGCATGAGCAAAGCGATGCCA | | 105 |
| | GATCCTGGGACCTGG | | |
| Da878 | TTTGCTCATGCGGTGAAAGTGATAGTAAC | | 106 |

Sulfotransferase-containing pBad plasmids for initial screening were constructed by Gibson Assembling of pBad vector amplified from pBad-BER2-ScFv with Da343&344 and sulfotransferase fragment amplified from synthetic DNA. pEvol-NnSULT1C1 was acquired by Gibson Assembling of pEvol-Mj vector amplified by Da443&444 and NnSULT1C1 amplified from pBad-NnSULT1C1 with Da441&442. To generate pEvol-cysDNC, cysDNC cassette was amplified from E. coli genome with Da446 and Da447 and inserted into pEvol vector amplified from pEvol-Mj with Da443&445. To generate pEvol-cysDNCQ, cysDNC cassette and cysQ cassette amplified separately from E. coli genome with Da446&448 and Da449&450, respectively, were overlapped and Gibson Assembled into pEvol vector amplified from pEvol-Mj with Da443&445. pEvol-NnSULT1C1-cysDNC(Q) was generated by inserting NnSULT1C1 fragment amplified from pBad-NnSULT1C1 into the vector of pEvol-cysDNC(Q) amplified with Da444&454.

To generate the plasmid for sulfotyrosine genetic incorporation in mammalian cells, OMeYRS of pAcBac2.tR4-OMeYRS/GFP* was substituted with sulfotyrosine-selective EcTyrRS mutant (L71V, D182G, L186M), which was achieved by Gibson Assembling of synthetic sulfotyrosineRS amplified with Da462&463 and pAcBac2.tR4-OMeYRS/GFP* vector digested by Xho1 and Nhe1. To integrate NnSULT1C1 into genome of HEK293T, PB-NnSULT1C1 was constructed by Gibson assembling synthetic NnSULT1C1 amplified by Da655&656 and Piggybac vector digested with BsrG1.

pET22b-T5-chi was constructed by Gibson Assembling of synthetic chimadanin sequence amplified with Da556&557 and pET22b-T5-sfGFP151TAG vector digested with Hind3 and Nde1. pET22b-T5-chi-28TAG and pET22b-T5-chi-31TAG were made according to the protocol on NEBaseChanger with Da664&665 and Da584&585, respectively, using pET22b-T5-chi as their template. pET22b-T5-chi-28TAG31TAG were made by following the same protocol with Da666&Da664 with pET22b-T5-chi-31TAG as a template. pET22b-T5-mad was constructed by Gibson Assembling of synthetic madanin-2 sequence amplified with Da558&559 and pET22b-T5-sfGFP151TAG vector digested with Hind3 and Nde1. pET22b-T5-mad-32TAG and pET22b-T5-mad-35TAG were made according to the protocol on NEBaseChanger with Da586&587 and Da661&662 with pET22b-T5-mad as a template. pET22b-T5-mad-32TAG35TAG were made by following the same protocol with Da663&Da661 using pET22b-T5-mad-32TAG as a template.

To explore whether sulfotransferase with similar structure with NnSULT1C1 could catalyze tyrosine sulfation, pEvol-mSULT1D1/hSULT1C2-cysDNCQ was constructed by assembling synthetic sulfotransferases amplified by Da832&833/Da838&839 with pEvol-NnSULT1C1-cysDNCQ vector amplified with Da802&803. To test the importance of NnSULT1C1 loop (SIQEPPAAS) and residues (T30, 133, W93, E161) involved in substrate binding, their corresponding mutants was obtained by Gibson Assembling pEvol-NnSULT1C1-cysDNCQ fragments amplified with Da858&859, Da871&872, Da873&874, Da875&876, Da877&878. To express NnSULT1C1 with a his6 tag at C terminal for its kinetics measurement, NnSULT1C1 was amplified from pEvol-NnSULT1C1-cysDNCQ with Da687&688 and Gibson Assembled with pET22b-T5-sfGFP151TAG digested with Hind3 and Nde1, which yields pET22b-T5-NnSULT11-His6.

Expression and Purification of Proteins: E. coli BL21(DE3) cells, transformed with pUltra-sulfotyrosineRS, pLei-sfGFP134TAG, and pBad-Empty/pBad-HsSULT1A1/pBad-HsSULT1A3/pBad-RnSULT1A1/pBad-GgSULT1C1/pBad-CsSULT1C2, were grown in 2YT medium at 37° C. The protein expression was carried out in Luria-Bertani (LB) medium with or without 1 mM sulfotyrosine addition. When the OD600 of the cell culture reached 0.6, protein expression was induced by the addition of IPTG and l-arabinose to a final concentration of 1 mM and 0.2%, respectively. After growth overnight at 30° C., cells were harvested by centrifugation at 4,750×g for 10 min and used for GFP fluorescence and cell optical density measurements. (FIG. 6) BW25113, ΔtrpE BW25113, ΔtyrA BW25113, ΔackA BW25113, ΔptsH BW25113, ΔcysH BW25113 cells were transformed with pUltra-sulfotyrosineRS, pET22b-T5-sfGFP151TAG, and pEvol-NnSULT1C1/pEvol-Empty, were grown in 2YT medium at 37° C. The protein expression was carried out in Luria-Bertani (LB) medium with or without 1 mM sulfotyrosine addition. When the OD600 of the cell culture reached 0.6, protein expression was induced by the addition of IPTG and l-arabinose to a final concentration of 1 mM and 0.2%, respectively. After growth overnight at 30° C., cells were harvested by centrifugation at 4,750×g for 10 min and used for GFP fluorescence and cell optical density measurements. (FIG. 3B)

ΔcysH BW25113, transformed with pUltra-sulfotyrosineRS, pET22b-T5-sfGFP151TAG, and pEvol-Empty/pEvol-NnSULT1C1/pEvol-NnSULT1C1-cysDNC/pEvol-NnSULT1C1-cysDNCQ, were grown in 2YT medium at 37° C. The protein expression was carried out in Luria-Bertani (LB) medium with or 1 mM sulfotyrosine addition. When the OD600 of the cell culture reached 0.6, protein expression was induced by the addition of IPTG and 1-arabinose to a final concentration of 1 mM and 0.2%, respectively. After growth overnight at 30° C. cells were harvested by centrifugation at 4,750×g for 10 min and used for GFP fluorescence and cell optical density measurements. (FIG. 3C) ΔcysH BW25113 cells, transformed with pUltra-sulfotyrosineRS, pET22b-T5-sfGFP151TAG, and pEvol-Empty/pEvol-NnSULT1C1-cysDNCQ, were grown in 2YT medium at 37° C. The protein expression was carried out in Luria-Bertani (LB) medium. When the OD600 of the cell culture reached 0.6, NnSULT1C1 expression was induced by indicated concentration of l-arabinose and grown at 30° C. for 6 h. Then the cells were diluted 5 times to OD 0.6. Expression of reporter sfGFP and sulfotyrosineRS were induced with 1 mM IPTG and indicated concentration of sulfotyrosine was added at same time. Additional l-arabinose was also added to maintain its indicated concentration. After growth at 30° C. for 18 hours, cells were harvested by centrifugation at 4,750×g for 10 min and used for GFP fluorescence and cell optical density measurements. (FIG. 3D) Proteins were purified on Ni-NTA resin (Qiagen). The purified protein was used for SDS-PAGE and ESI-MS analysis. (FIGS. 3F-H)

ΔcysH BW25113 cells, transformed with pUltra-sulfotyrosineRS, pET22b-T5-sfGFP151TAG, and pEvol-Empty/pEvol-NnSULT1C1-cysDNCQ, were grown in 2YT medium at 37° C. The protein expression was carried out in Luria-Bertani (LB) medium. When the OD600 of the cell culture reached 0.6, NnSULT1C1 expression was induced by 15 mg/L l-arabinose and grown at 30° C. for 6 h. Then the cells were diluted 5 times to OD 0.6. Expression of reporter sfGFP and sulfotyrosineRS were induced with 1 mM IPTG and indicated concentration of sulfotyrosine was added at same time. Additional l-arabinose was also added to maintain its final concentration of 15 mg/L. After growth overnight at 30° C. for 18 hours, cells were harvested by centrifugation at 4,750×g for 10 min and used for measuring cellular sulfotyrosine concentration. (FIG. 3E)

To express wildtype thrombin inhibitors, BL21(DE3) cells transformed with either pET22b-T5-chi or pET22b-T5-mad were grown in 2YT medium at 37° C. The protein expression was carried out in LB medium. When the OD600 of the cell culture reached 0.6, protein expression was induced by the addition of 0.4 mM IPTG. After growth overnight at 18° C. for 18 hours, cells were harvested by centrifugation at 4,750×g for 10 min. Proteins were purified on Ni-NTA resin (Qiagen). The purified protein was used for SDS-PAGE and ESI-MS analysis. (FIG. 5C)

To express thrombin inhibitors containing sulfotyrosine, ΔcysH BW25113 cells, transformed with pUltra-sulfotyrosineRS, pET22b-T5-inhibitor-X-TAG, and pEvol-NnSULT1C1-cysDNCQ, were grown in 2YT medium at 37° C. In the control group, ΔcysH BW25113 cells were transformed with pUltra-sulfotyrosineRS, pET22b-T5-inhibitor-X-TAG, and pEvol-Empty. When the OD600 of the cell culture reached 0.6, NnSULT1C1 expression was induced by 15 mg/L concentration of l-arabinose and grown at 30° C. for 6 h. Then the cells were diluted 5 times to OD 0.6. Expression of inhibitor and sulfotyrosineRS were induced with 1 mM IPTG and 3 mM sulfotyrosine was added to only control cells at same time. Additional l-arabinose was also added to maintain its final concentration of 15 mg/L. After growth overnight at 18° C. for 18 hours, cells were harvested by centrifugation at 4,750×g for 10 min. Proteins were purified on Ni-NTA resin (Qiagen) following the manufacturer's instructions. The purified protein was used for SDS-PAGE and ESI-MS analysis. (FIG. 5C and FIG. 16)

To test the importance of the variable loop and residues in binding pockets, ΔcysH BW25113 cells, transformed with pUltra-sulfotyrosineRS, pET22b-T5-sfGFP151TAG, and pEvol-NnSULT1C1-cysDNCQ with indicated sequence mutations were grown in 2YT medium at 37° C. To test the tyrosine sulfation activity of the top 3 sulfotransferases with similar structure to NnSULT1C1, ΔcysH BW25113 cells, transformed with pUltra-sulfotyrosineRS, pET22b-T5-sfGFP151TAG, and pEvol-X sulfotransferase-cysDNCQ were grown in 2YT medium at 37° C. The protein expression was carried out in LB medium. When the OD600 of the cell culture reached 0.6, sulfotransferase expression was induced by 15 mg/L l-arabinose and grown at 30° C. for 6 h. Then the cells were diluted 5 times to OD 0.6. Expression of sfGFP and sulfotyrosineRS were induced with 1 mM IPTG. Additional l-arabinose was also added to maintain its final concentration of 15 mg/L. After growth overnight at 30° C. for 18 hours, cells were harvested by centrifugation at 4,750×g for 10 min and used for GFP fluorescence and cell optical density measurements. (FIGS. 2B, 2C, and 2F)

To express NnSULT1C1 for kinetics measurement, BL21(DE3) cell transformed with pET22b-T5-NnSULT11-His6 was grown in 2YT medium at 37° C. The protein expression was carried out in Luria-Bertani (LB) medium. When the OD600 of the cell culture reached 0.6, protein expression was induced by the addition of 0.3 mM IPTG. After growth overnight at 30° C. for 18 hours, cells were harvested by centrifugation at 4,750×g for 10 min. Proteins were purified on Ni-NTA resin (Qiagen) following the manufacturer's instructions. The purified protein was used for SDS-PAGE and kinetic assay. (FIG. 12)

Expression and Fluorescence Measurement of sfGFP: After sfGFP expression with the methods described above, 0.5 mL cells were harvested by centrifugation at 4,750×g for 10 min and then suspended with 0.5 ml PBS (pH 7.4). Fluorescence of cells was measured using excitation/emission wavelengths of 395/509 nm. Optical Density at 600 nm was also obtained. The sfGFP fluorescence/OD600 was used as the normalized fluorescence. The error bars represent the standard deviations of 3 independent protein expression trials.

E. coli Intracellular sulfotyrosine Concentration Measurement: Cells were harvested by centrifugation at 4,750×g for 10 min and washed with PBS 7.4 for three times. The cell pellets were re-suspended in 300 μL of bugbuster lysis buffer: toluene (80:20) solution and shaken at 30° C. for 1 h. The resulting lysate was centrifuged at 21000 g for 30 min at 4° C. 200 μl supernatant was transferred to a new tube and re-centrifuged at 21000 g for 2 h. 50 μl supernatant from the top was then analyzed using the LC-MS. An Agilent 1260 Infinity II LC System coupled with Single Quadrupole ESI-MS System was used for analysis of all samples. To measure the sulfotyrosine ions, ions detected were set to selected ion monitoring (SIM) mode (262 m/z) to detect positive ions of sulfotyrosine. Standards of 1 μM, 25 μM, 50 μM, 100 μM, 200 μM, and 400 μM of authentic sulfotyrosine (Bachem) were also prepared and analyzed by the same method. Using LC-MS data, a linear standard curve was generated based on peak areas corresponding to sulfotyrosine ions and the concentration of sulfotyrosine in standards. The standard curve was then used to calculate the concentrations of sulfotyrosine from different cell lysates. Each sample was carried out in n=3 independent groups. The intracellular concentration of sulfotyrosine in cells was calculated based on the following equation.

[sTyr intracellular]=(sTyr concentration in lysate×volume of lysate)/total cell numbers×E. coli cell volume

Total cell numbers were calculated with the approximate values: 8×10⁸ cells per OD600. 0.6 fL was used as an average E. coli cell volume.

Exploration on evolutionary relationship of NnSULT1C1: The rooted phylogenetic tree was inferred using the UPGMA method in MEGA X software. The UPGMA algorithm constructs the tree that reflects the genetic distance between protein sequences present in a pairwise similarity matrix. The tree is drawn to scale, with branch lengths in the same units as those of the evolutionary distances used to infer the phylogenetic tree. The evolutionary distances were computed using the Poisson correction method and are in the units of the number of amino acid substitutions per site. 10 sequences from bottom branch were used for Multiple sequence alignment (MSA) with Maftt method. The alignment result was visualized in Jalview software. The sequence consensus was analyzed as shown in FIG. 7.

Kinetics Measurement of NnSULT1C1: The purified NnSULT1C1 was buffer exchanged to 10 mM NH4OAc buffer pH 8 via PD-10 column. The concentration of enzyme was calculated based on its Absorption at 280 nm. The enzymatic reaction was performed in 100 μL 10 mM NH4OAc buffer pH 8 including 1 μM NnSULT1C1, 20 μM PAPS, 5 mM CaCl₂) and variable concentration of tyrosine. The reactions were incubated at 37° C. and quenched with 100 μL ACN at 5 minutes. The supernatants of these mixtures were used for sulfotyrosine quantification via ESI-MS. To improve the sensitivity of sulfotyrosine detection, Selected Ion Monitoring (SIM) mode was used to detect the negative ion (m/z=260) under 50° C. drying gas temperature and 2400 V capillary voltage. The standard curve of authentic sulfotyrosine was prepared under the same condition, which yields the linear relationship between area under the curve and its sulfotyrosine concentration. To obtain the concentration of sulfotyrosine produced in enzymatic reaction, its area under the curve was used to calculate sulfotyrosine concentration based on the equation obtained from standard curve. Each sample was carried out in n=3 independent samples. The data was fitted to Michaelis-Menten equation in Prism.

Protein Purification from Mammalian Cells: To confirm the genetic incorporation of sulfotyrosine from either biosynthesis or external addition, HEK293T and HEK293T-NnSULT1C1 cells were transfected with pAcBac2.tR4-sulfotyrosineRS/GFP* with Polyjet In vitro DNA Transfection Reagent (SignaGen Laboratories) in the presence or absence of 3 mM sulfotyrosine addition. Mediums were changed at 12-16 hour after transfection. After 48 hours of transfection, cells were harvested with trypsin and subsequently washed by DPBS 3 times. Cells were lysed using the Mammalian Cell PE LB reagent (G-Bioscience) according to its manual. The cell lysates were centrifuged at 15,000 rpm for 10 minutes. The protein in the supernatant was purified from the supernatant using Ni-NTA resin (Qiagen) following the manufacturer's instruction. The purified protein was used for ESI-MS analysis.

Mammalian Cell sulfotyrosine Concentration Measurement: HEK293 T and HTEK293 T-NnSULT1C1 were detached from plate with trypsin and washed with DPBS for 3 times. The number of cells was counted by hemocytometer. Cells were resuspended in 0.5 mL methanol-water (2:3) and lyzed by six freeze-thaw cycles. The resulting cell lysates were centrifuged at 21000×g for 1 h at 4° C. The resulting supernatants were injected to LC-MS for the quantification of sulfotyrosine with selected ion monitoring (SIM) mode. An Agilent 1260 Infinity II LC System coupled with Single Quadrupole ESI-MS System was used for the analysis of all samples. To measure the sulfotyrosine ions, ions detected were set to Selected Ion Monitoring (SIM) mode (262 m/z) to detect positive ions of sulfotyrosine. Standards of 1 μM, 25 μM, 50 μM, 100 μM, 200 μM, and 400 μM of purchased sulfotyrosine (Bachem) dissolved in methanol-water (2:3) were also prepared and analyzed by the same method. A linear standard curve was generated based on peak areas corresponding to sulfotyrosine ions and the concentration of sulfotyrosine in standard samples. The standard curve was then used to calculate the concentration of sulfotyrosine from different cell lysates. The intracellular concentration of sulfotyrosine in cells was calculated with the following equation.

[sTyr intracellular]=(sTyr concentration in lysate×volume of lysate)/total cell numbers×E. coli cell volume

2 μL was used as an average volume of mammalian cells.

Mass Spectra Methods for Proteins: A single quadrupole mass spectrometer (Agilent: G7129A) coupled with 1260 infinity II Quaternary Pump (Agilent: G7111B) was used for all the protein samples with PLRP-S(1000A, 5 μm) column. Water with 0.1% formic acid and ACN with 0.1% formic acid were the organic and aqueous mobile phase, respectively. Flow gradient was initially set at 5% ACN, 15% ACN at 0.1 min, 55% ACN at 4.5 min and then back to 10% ACN at 5 min. Spectra were deconvoluted using the Maximum Entropy deconvolution algorithm in the software BioConfirm.

### REFERENCES

The following references, to the extent that they provide exemplary procedural or other details supplementary to those set forth herein, are specifically incorporated herein by reference.
Allali-Hassani et al., PLOS Biol., 5:97, 2007.
Ambrogelly et al., Nat. Chem. Biol., 3:29-35, 2007.
Badri et al., Biotechnol. J, 14:1800436, 2019.
Bang et al., Microb. Cell Factories, 15:16, 2016.
Banks et al., J. Comput. Chem., 26:1752-1780, 2005.
Berger et al., PLOS ONE, 6:26794, 2011.
Bidwell et al., J. Mol. Biol., 293:21-530, 1999.
Blanchard et al., Pharmacogenetics, 14:199-211, 2004.
Bundy & Swartz, Bioconjug. Chem., 21:255-263, 2010.
Burkovski & Kramer, Appl. Microbiol. Biotechnol., 58:265-274, 2002.
Chatterjee et al., Proc. Natl. Acad. Sci., 110:11803-11808, 2013.
Chen et al., ACS Chem. Biol., 14:2793-2799, 2019.
Chen et al., Chem., 6:2717-2727, 2020.
Chen et al., Chem. Commun., 54:7187-7190, 2018.
Chen et al., J. Am. Chem. Soc., 135:14940-14943, 2013.
Chen et al., J. Mol. Biol., 167412, 2021.
Chen et al., Nat. Chem. Biol., 18:47-55, 2022.
Chin, Annu. Rev. Biochem., 83:379-408, 2014.
Chin, Nature, 550:53-60, 2017.
Choe et al., Cell, 114:161-170, 2003.
Chu et al., Front. Microbiol., 9, 2018.
Copp et al., Biochem., 57:4651-4662, 2018.
Corral-Rodriguez et al., J. Med. Chem., 53:3847-3861, 2010.
De la Torre & Chin, Nat. Rev. Genet., 22:169-184, 2021.
Dien et al., Curr. Opin. Chem. Biol., 46:196-202, 2018.
Farzan et al., Cell, 96:667-676, 1999.
Friesner et al., J. Med. Chem., 47:1739-1749, 2004.
Gamage et al., Toxicol. Sci., 90:5-22, 2006.
Gerlt et al., Biochim. Biophys. Acta, 1854:1019-1037, 2015.
Guo & Niu, J. Mol. Biol., 167346, 2021.
He et al. Nat. Commun., 11:4820, 2020.
Hirschmann et al., Front. Plant Sci., 5, 2014.
Hoppmann et al., Nat. Chem. Biol., 13:842-844, 2017.
Hsieh et al., Angew. Chem. Int. Ed Engl., 53:3947-3951, 2014.
Huang & Liu, Synth. Syst. Biotechnol., 3:150-158, 2018.
Iannuzzelli & Fasan, Chem. Sci., 11:6202-6208, 2020.
Italia et al., Nat. Chem. Biol., 16:379-382, 2020.
Jendresen & Nielsen, Nat. Commun., 10:1-10, 2019.
Jin et al., IUCrJ, 2020.
Jin et al., J. Chem. Theory Comput., 16:3977-3988, 2020.
Jumper et al., Nature, 596:583-589, 2021.
Kazimirovi & Stibriniovi, Front. Cell. Infect. Microbiol., 3, 2013.
Ko et al., ACS Synth. Biol., 8:1195-1203, 2019.
Koh & Kini, Thromb. Haemost., 102:437-453, 2009.
Lee et al., Science, 326:850-853, 2009.
Li et al., Biochem., 57:2903-2907, 2018.
Liu & Schultz, Annu. Rev. Biochem., 79:413-444, 2010.
Liu & Schultz, Nat. Biotechnol., 24:1436-1440, 2006.
Liu et al. Proc. Natd. Acad. Sci., 105:17688-17693, 2008.
Liu et al., Biochem., 48:8891-8898, 2009.
Liu et al., Nat. Protoc., 4:1784-1789, 2009.
Lu et al., Biochem. Biophys. Res. Commun., 335:417-423, 2005.
Ludeman, & Stone, Br. J. Pharmacol., 171:1167-1179, 2014.
Luo, X. et al. Nat. Chem. Biol., 13:845-849, 2017.
Mehl et al., J. Am. Chem. Soc., 125:935-939, 2003.
Moore, Proc. Natd. Acad. Sci., 106:14741-14742, 2009.
Nakajima et al., J Vet. Med. Sci., 68:447-452, 2006.
Negishi et al., Arch. Biochem. Biophys. 390(2):149-57, 2001.
Neuwald et al., J Bacteriol., 174:415-425, 1992.
Owens et al., ACS Cent. Sci., 6:368-381, 2020.
Palacin et al., Physiol. Rev., 78:969-1054, 1998.
Rath et al., Drug Discov. Today. 9(23):1003-11, 2004.
Rogerson et al., Nat. Chem. Biol., 11:496-503, 2015.
Schlee, Syst. Zool., 24:263-268, 1975.
Schwessinger et al., Integr. Biol., 8:542-545, 2016.
Seibert & Sakmar, Biopolymers, 90:459-477, 2008.
Somers et al., Cell, 103:467-479, 2000.
Spiegelberg et al., J. Biol. Chem., 274:13619-13628, 1999.
Suiko et al., Biosci. Biotechnol. Biochem., 81:63-72, 2017.
Tanaka-Azevedo et al., J Biomed. Biotechnol., 2010.
Thompson et al., Nat. Chem., 9:909-917, 2017.
Tunyasuvunakool et al., Nature, 596:590-596, 2021.
Varin et al., FASEB J, 11:517-525, 1997.
Veldkamp et al., Sci. Signal., 1:ra4, 2008.
Wang et al., Annu. Rev. Biophys. Biomol. Struct., 35:225-249, 2006.
Wang et al., Science, 292:498-500, 2001.
Watson et al., ACS Cent. Sci., 4:468-476, 2018.
Watson et al., Proc. Natd. Acad. Sci., 116:13873-13878, 2019.
Wei et al., Sci. Rep., 6, 2016.
Westmuckett et al., PLOS ONE, 6:20406, 2011.
Xiao & Schultz, Cold Spring Harb. Perspect. Biol., a023945, 2016.
Yang et al., Molecules, 20:2138-2164, 2015.
Young & Schultz, ACS Chem. Biol., 13:854-870, 2018.
Yusa et al., Proc. Natd. Acad. Sci., 108:1531-1536, 2011.
Zhang & Ai, Nat. Chem. Biol., 16:1434-1439, 2020.
Zhang et al., Nat. Methods, 14:729-736, 2017.
Zhu et al., Appl. Environ. Microbiol., 80:3072-3080, 2014.

1. An engineered cell comprising a sulfotransferase having a sequence that is at least about 95% identical to the amino acid sequence of SEQ ID NO: 1.

2. (canceled)

3. The engineered cell of claim 1, wherein the sulfotransferase is a tyrosine sulfotransferase.

4. The engineered cell of claim 1, wherein the engineered cell further comprises a tyrosyl-tRNA synthetase/tRNA pair.

5. The engineered cell of claim 4, wherein the tyrosyl-tRNA synthetase/tRNA pair is derived from E. coli.

6. The engineered cell of claim 1, wherein the engineered cell further comprises 3′-phosphoadenosine-5′-phosphosulfate.

7. The engineered cell of claim 1, wherein the engineered cell further comprises a peptide comprising sulfotyrosine at one or more positions.

8. The engineered cell according to claim 7, wherein the peptide comprises sulfotyrosine at two or more positions.

9.-20. (canceled)

21. The engineered cell of claim 1, wherein the peptide is a thrombin-inhibitor.

22. The engineered cell of claim 1, wherein the engineered cell is a mammalian cell or a prokaryotic cell.

23. (canceled)

24. The engineered cell of claim 22, wherein the cell is an E. coli cell.

25. The engineered cell of claim 24, wherein the cell further expresses ATP sulfurylase, adenosine 5′-phosphosulfate kinase, and adenosine-3′,5′-diphosphate nucleotidase.

26. The engineered cell of claim 1, wherein the engineered cell is a eukaryotic cell.

27. The engineered cell of claim 26, wherein the engineered cell is a human embryonic kidney (HEK) cell.

28. (canceled)

29. The engineered cell of claim 1, wherein the cellular concentration of sulfotyrosine is greater than or equal to 500 μM.

30.-31. (canceled)

32. A method of expressing a recombinant peptide comprising at least one sulfotyrosine residue at a selected position not found in a wild-type version of the peptide, the method comprising:
a. obtaining an engineered cell that comprises 3′-phosphoadenosine-5′-phosphosulfate and a sulfotransferase that is at least about 95% identical to SEQ ID NO: 1;
b. expressing a nucleic acid encoding the recombinant peptide in the cell; and
c. purifying the recombinant peptide from the cell.

33. The method of claim 32, wherein the cell comprises an expression construct encoding the sulfotransferase operably linked to an arabinose-inducible promoter, wherein the method further comprises inducing sulfotransferase expression with L-arabinose.

34.-36. (canceled)

37. The method of claim 32, wherein the cellular concentration of sulfotyrosine is greater than or equal to 50 μM.

38.-40. (canceled)

41. A composition comprising the purified recombinant peptide produced by the method of claim 32.

42. The composition of claim 41, wherein at least 80% of the recombinant peptides in the composition comprise the sulfotyrosine residue at the selected position.

43.-51. (canceled)

52. The composition of claim 41 for use in the treatment or prevention of a disease or disorder.

53.-54. (canceled)
